(19) United States
(12) Patent Application Publication
(10) Pub. No.: US 2009/0018130 A1
(43) Pub. Date: Jan. 15, 2009

(54) Derivatives of 5-Aryl-1H-Pyrrolo [2, 3B] Pyridine-3-Carboxamide or 5-Aryl-1H-Pyrrolo [2, 3B] Pyridine-3-Carboxylic Acid

(76) Inventors: Stefan Berg, Sodertalje (SE); Johan Hedstrom, Sodertalje (SE); Sven Hellberg, Sodertalje (SE); Peter Soderman, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 11/571,183

(22) PCT Filed: Jun. 20, 2005
(86) PCT No.: PCT/SE05/00955

§ 371 Date: Oct. 15, 2007

(30) Foreign Application Priority Data
Jun. 24, 2004 (SE) 0401655-6

(51) Int. Cl. A61K 31/5377; C07D 471/04; A61K 31/437; A61K 31/496

(52) U.S. Cl. 514/234.5; 546/113; 514/300; 544/362; 514/253.04; 544/127

(57) ABSTRACT

The present invention relates to new compounds of formula I as a free base or a pharmaceutically acceptable salt, solvate or solvate of salt thereof, a process for their preparation and new intermediates used therein, pharmaceutical formulations containing said therapeutically active compounds and to the use of said active compounds in therapy.

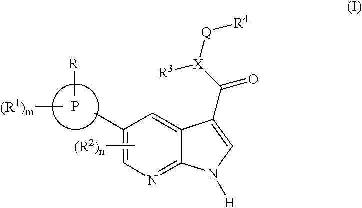

The present invention relates to new compounds of formula I, as a free base or a pharmaceutically acceptable salt, solvate or solvate of salt thereof, to pharmaceutical formulations containing said compounds and to the use of said compounds in therapy. The present invention further relates to a process for the preparation of compounds of formula I and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β) or Tau (τ) phosphorylating kinase selectively phosphorylates the microtubule associated protein τ in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Imahori and Uchida, J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

### Schizophrenia

GSK3 is involved in signal transduction cascades of multiple cellular processes, particularly during neural development. Kozlovsky et al (Am J Psychiatry 2000 May; 157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

### Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25; 95 (5):605-14)). The new follicles formed sebaceous glands and dermal papilla, normally established only in embryogenesis. Thus GSK3 inhibition may offer treatment for baldness.

### Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### Bone-Related Disorders

It has been shown that GSK3 inhibitors could be used for treatment of bone-related disorders. This has been discussed in e.g. Tobias et al., Expert Opinion on Therapeutic Targets, February 2002, pp 41-56.

### DISCLOSURE OF THE INVENTION

The object of the present invention is to provide compounds having a selective inhibiting effect at GSK3 as well as having a good bioavailability. Accordingly, the present invention provides a compound of the formula I:

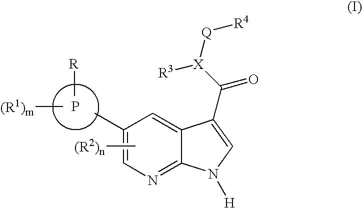

wherein:

P is phenyl or a 5- or 6-membered heteroaromatic ring containing one or more heteroatoms selected from N, O or S and said phenyl or 5- or 6-membered heteroaromatic ring may optionally be fused with a 5- or 6-membered saturated, partially saturated or unsaturated ring containing one or more atoms selected from C, N, O or S;

Q is independently selected from C₁₋₆alkyl, C₂₋₆alkenyl or C₂₋₆alkynyl, or Q is absent;

X is independently selected from N or O and when X is O, Q and R⁴ are absent;

R is independently selected from hydrogen, CN, NO₂, OH, NH₂, COOH, CONH₂, COCH₃, halogen, C₁₋₆alkyl, C₁₋₆alkoxy, fluoromethyl, difluoromethyl, trifluoromethyl, fluoromethoxy, difluoromethoxy, trifluoromethoxy;

R¹ is independently selected from C₁₋₆alkylC₃₋₆cycloalkyl, OR⁵, SR⁵, NR⁶R⁷, CO₂R⁵, COR⁵, (SO₂)R⁵, (SO)R⁵, (SO₂)NR⁶R⁷, NR⁸(SO₂)R⁵, CONR⁶R⁷, NR⁸COR⁵, NR⁸CONR⁶R⁷, NR⁸CO₂R⁵, (SO)NR⁶R⁷, NR⁸(SO)R⁵, C₁₋₆alkylOR⁵, C₁₋₆alkylSR⁵, C₁₋₆alkylNR⁶R⁷, C₁₋₆alkylCO₂R⁵, C₁₋₆alkylCOR⁵, C₁₋₆alkyl(SO₂)R⁵, C₁₋₆alkyl(SO)R⁵, C₁₋₆alkyl(SO₂)NR⁶R⁷, C₁₋₆alkylNR⁸(SO₂)R⁵, C₁₋₆alkylCONR⁶R⁷, C₁₋₆alkylNR⁸COR⁵, C₁₋₆alkylNR⁸CONR⁶R⁷, C₁₋₆alkylNR⁸CO₂R⁵, C₁₋₆alkyl(SO)NR⁶R⁷ or C₁₋₆alkylNR⁸(SO)R⁵; and wherein said C₁₋₆alkyl group is optionally substituted by one or more A;

R² is independently selected from hydrogen, C₁₋₆alkyl, CN, nitro, halogen, OR¹³, fluoromethyl, difluoromethyl, trifluoromethyl, fluoromethoxy, difluoromethoxy and trifluoromethoxy;

R³ is independently selected from hydrogen, C₁₋₆alkyl, C₂₋₆alkenyl, C₂₋₆alkynyl and C₀₋₆alkylC₃₋₆cycloalkyl, wherein said C₁₋₆alkyl, C₂₋₆alkenyl, C₂₋₆alkynyl and C₀₋₆alkylC₃₋₆cycloalkyl group is optionally substituted by one or more A;

R⁴ is independently selected from hydrogen, halogen, nitro, CHO, CN, OC₁₋₆alkylCN, OR¹³, OC₁₋₆alkylOR¹³, fluoromethyl, difluoromethyl, trifluoromethyl, fluoromethoxy, difluoromethoxy, trifluoromethoxy, NR¹³R¹⁴, OC₁₋₆alkylNR¹³R¹⁴, NR¹³OR¹⁴, CO₂R¹³, OC₁₋₆alkylCO₂R¹³, CONR¹³R¹⁴, OC₁₋₆alkylCONR¹³R¹⁴, OC₁₋₆alkylNR¹³(CO)R¹⁴, NR¹³(CO)R¹⁴, O(CO)NR¹³R¹⁴, NR¹³(CO)OR¹⁴, NR¹³(CO)NR¹³R¹⁴, O(CO)OR¹³, O(CO)R¹³, COR¹³, OC₁₋₆alkylCOR¹³, NR¹³(CO)(CO)R¹⁴, NR¹³(Co)(CO)NR¹³R¹⁴, SR¹³, (SO₂)NR¹⁴R¹³, OC₁₋₆alkylNR¹³(SO₂)R¹⁴, NR⁵(SO₂)R¹³, OC₀₋₆alkyl(SO₂)NR¹³R¹⁴, (SO)NR¹³R¹⁴, OC₁₋₆alkyl(SO)NR¹³R¹⁴, SO₃R¹³, NR⁵(SO₂)NR¹³R¹⁴, NR¹³(SO)R¹⁴, OC₁₋₆alkylNR¹³(SO)R¹⁴, OC₀₋₆alkylSO₂R¹³, SO₂R¹³, SOR¹³, C₃₋₆cycloalkyl, aryl, a 5- or 6-membered heteroaromatic ring containing one or more heteroatoms independently selected from N, O, or S, a 5- or 6-membered heterocyclic ring containing one or more heteroatoms independently selected from N, O, or S, which heterocyclic group may be saturated or unsaturated, and wherein any said C₃₋₆cycloalkyl, aryl, 5- or 6-membered heteroaromatic ring or a 5- or 6-membered heterocyclic ring is optionally substituted by one or more A, or R⁴ may be absent;

R⁵ is independently selected from C₃₋₆cycloalkyl, C₁₋₆alkylC₃₋₆cycloalkyl, C₂₋₆alkylNR⁹R¹⁰, C₂₋₆alkylOC₂₋₆alkylNR⁹R¹⁰, C₂₋₆alkyl(SO₂)NR⁹R¹⁰, C₂₋₆alkyl(SO)NR⁹R¹⁰, C₂₋₆alkylNR¹¹(SO)R¹², C₂₋₆alkylNR⁹(SO₂)R¹⁰, C₂₋₆alkyl(SO₂)C₂₋₆alkylNR⁹R¹⁰, C₂₋₆alkyl(SO)C₂₋₆alkylNR⁹R¹⁰, C₂₋₆alkylSC₂₋₆alkylNR¹¹R¹², C₂₋₆alkylCONR⁹R¹⁰, C₂₋₆alkylNR⁹COR¹⁰, heteroaryl or C₁₋₆alkylheteroaryl wherein any C₂₋₆alkyl or heteroaryl is optionally substituted by one or more A;

R⁶ is independently selected from C₂₋₆alkenyl, C₂₋₆alkynyl, C₀₋₆alkylC₃₋₆cycloalkyl, C₁₋₆alkylNR⁹R¹⁰, C₁₋₆alkylOC₁₋₆alkylNR⁹R¹⁰, C₀₋₆alkyl(SO₂)NR⁹R¹⁰, C₁₋₆alkyl(SO)NR⁹R¹⁰, C₁₋₆alkylNR¹¹(SO)R¹², C₁₋₆alkylNR⁹(SO₂)R¹⁰, C₀₋₆alkyl(SO₂)C₁₋₆alkylNR⁹R¹⁰, C₁₋₆alkyl(SO)C₁₋₆alkylNR⁹R¹⁰, C₂₋₆alkylSC₂₋₆alkylNR¹¹R¹², C₁₋₆alkylCONR⁹R¹⁰, C₀₋₆alkylNR⁹COR¹⁰, C₁₋₆alkylaryl or C₀₋₆alkylheteroaryl wherein any C₁₋₆alkyl, C₂₋₆alkenyl, C₂₋₆alkynyl, C₀₋₆alkylC₃₋₆cycloalkyl, C₁₋₆alkylaryl, C₀₋₆alkylheteroaryl is optionally substituted by one or more A;

R⁷ is independently selected from hydrogen and C₁₋₆alkyl;

R⁶ and R⁷ may together form a 5- or 6-membered heterocyclic ring containing one or more heteroatoms selected from N, O or S, wherein said heterocyclic ring is optionally substituted by one or more A;

R⁸ is independently selected from hydrogen, C₁₋₆alkyl, C₂₋₆alkenyl, C₂₋₆alkynyl, C₀₋₆alkylC₃₋₆cycloalkyl;

R⁹ is independently selected from C₁₋₆alkyl-B, C₂₋₆alkenyl, C₂₋₆alkynyl, C₃₋₆cycloalkyl, C₁₋₆alkylC₃₋₆cycloalkyl, aryl, C₁₋₆alkylaryl, heteroaryl, C₁₋₆alkylheteroaryl wherein any C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;

R¹⁰ is independently selected from hydrogen and C₁₋₆alkyl;

R⁹ and R¹⁰ may together form a 5- or 6-membered heterocyclic ring containing one or more heteroatoms selected from N, O or S, wherein said heterocyclic ring is optionally substituted by one or more A;

R¹¹ and R¹² are independently selected from hydrogen, C₁₋₆alkyl, C₂₋₆alkenyl, C₂₋₆alkynyl, C₃₋₆cycloalkyl, C₁₋₆alkylC₃₋₆cycloalkyl, aryl, C₁₋₆alkylaryl, heteroaryl, C₁₋₆alkylheteroaryl, wherein any C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;

R¹¹ and R¹² may together form a 5- or 6-membered heterocyclic ring containing one or more heteroatoms selected from N, O or S, wherein said heterocyclic ring is optionally substituted by one or more A;

R¹³ and R¹⁴ are independently selected from hydrogen, C₁₋₆alkyl, C₂₋₆alkenyl, C₂₋₆alkynyl, C₃₋₆cycloalkyl, C₁₋₆alkylC₃₋₆cycloalkyl, C₁₋₆alkylNR¹⁵R¹⁶, aryl, C₁₋₆alkylaryl, heteroaryl, C₁₋₆alkylheteroaryl, wherein any said C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;

R¹³ and R¹⁴ may together form a 5- or 6-membered heterocyclic ring containing one or more heteroatoms selected from N, O or S, wherein said heterocyclic ring is optionally substituted by one or more A;

R¹⁵ and R¹⁶ are independently selected from hydrogen and C₁₋₆alkyl;

R¹⁵ and R¹⁶ may together form a 5- or 6-membered heterocyclic ring containing one or more heteroatoms selected from N, O or S, wherein the said heterocyclic ring is optionally substituted by one or more A;

m is 1;

n is 1 or 2;

A is selected from: halogen, nitro, oxo (═O), CHO, CN, OR¹⁵, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR¹⁵R¹⁶, OC₁₋₆alkylNR¹⁵R¹⁶, CO₂R¹⁵, CONR¹⁵R¹⁶, NR¹⁵(CO)R¹⁶, O(CO)R¹⁵, COR¹⁵, SR¹⁵, (SO₂)NR¹⁵R¹⁶, (SO)NR¹⁵R¹⁶, SO₃R¹⁵, SO₂R¹⁵ or SOR¹⁵;

B is nitro, oxo (═O), CHO, CN, OR¹⁵, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR¹⁵R¹⁶, OC₁₋₆alkylNR¹⁵R¹⁶, CO₂R¹⁵, CONR¹⁵R¹⁶, NR¹⁵(CO)R¹⁶, O(CO)R¹⁵, COR¹⁵, SR¹⁵, (SO₂)NR¹⁵R¹⁶, (SO)NR¹⁵R¹⁶, SO₃R¹⁵, SO₂R¹⁵ or SOR¹⁵; as a free base or a pharmaceutically acceptable salt, solvate or solvate of a salt thereof.

In one aspect of the invention, there are provided compounds according to formula I, wherein P is phenyl.

In another aspect of the invention, there is provided compounds according to formula I, wherein R is selected from hydrogen, halogen, C₁₋₆alkyl, trifluoromethyl and trifluoromethoxy. In one embodiment of this aspect, there is provided compounds according to formula I, wherein R is selected from hydrogen and trifluoromethoxy.

In yet another aspect of the invention, there is provided compounds according to formula I, wherein R¹ is selected from NR⁶R⁷, C₁₋₆alkylNR⁶R⁷, CONR⁶R⁷, (SO₂)NR⁶R⁷ and OR⁵.

In yet another aspect of the invention, there is provided compounds according to formula I, wherein R² is hydrogen.

In yet another aspect of the invention, there is provided compounds according to formula I, wherein R³ is hydrogen or C₁₋₆alkyl. In one embodiment of this aspect, R³ is hydrogen.

In yet another aspect of the invention, there is provided compounds according to formula I, wherein R⁴ is independently selected from OR¹³, NR¹³R¹⁴, CN, SOR¹³, SO₂R¹³, and a 5- or 6-membered heteroaromatic ring containing one or more heteroatoms independently selected from N, O, or S, or a 5- or 6-membered heterocyclic ring containing one or more heteroatoms independently selected from N, O, or S which heterocyclic group may be saturated or unsaturated, and wherein said 5- or 6-membered heteroaromatic ring or said 5- or 6-membered heterocyclic ring is optionally substituted by one or more A. In one embodiment of this aspect, there is provided compounds according to formula I, wherein said A is selected from OR¹⁵ and SO₂R¹⁵.

In yet another aspect of the invention, there is provided compounds according to formula I, wherein R¹³ and R¹⁴ together form a 5- or 6-membered heterocyclic ring containing one or more heteroatoms selected from N, O or S, wherein said heterocyclic ring is optionally substituted by one or more A. In one embodiment of this aspect, there is provided compounds according to formula I, wherein said A is selected from C₁₋₆alkyl.

In yet another aspect of the invention, there is provided compounds according to formula I, wherein Q is C₁₋₆alkyl, or Q is absent.

In yet another aspect of the invention, there is provided compounds according to formula I, wherein X is N.

In yet another aspect of the invention, there is provided compounds according to formula I, wherein R is selected from hydrogen, and trifluoromethoxy; R¹ is selected from NR⁶R⁷, C₁₋₆alkylNR⁶R⁷, CONR⁶R⁷, (SO₂)NR⁶R⁷, and OR⁵; R² and R³ is hydrogen; R⁴ is independently selected from OR¹³, NR¹³R¹⁴, SO₂R¹³, and a 5- or 6-membered heteroaromatic ring containing one or more heteroatoms independently selected from N, O, or S, a 5- or 6-membered heterocyclic ring containing one or more heteroatoms independently selected from N, O, or S which heterocyclic group may be saturated or unsaturated, and wherein said 5- or 6-membered heteroaromatic ring or said 5- or 6-membered heterocyclic ring is optionally substituted by one or more A; R¹³ and R¹⁴ are independently selected from hydrogen, and C₁₋₆alkyl; R¹³ and R¹⁴ together form a 5- or 6-membered heterocyclic ring containing one or more heteroatoms selected from N, O or S, wherein the said heterocyclic ring is optionally substituted by a group selected from A; R¹⁵ is C₁₋₆alkyl; Q is C₁₋₆alkyl, or Q is absent; A is C₁₋₆alkyl, SO₃R¹⁵, or OR¹⁵.

In yet another aspect of the invention, there is provided compounds being:
N-(3-Methoxypropyl)-5-{4-[(4-methylpiperazin-1-yl)sulfonyl]phenyl}-1H-pyrrolo[2,3-b]pyridine-3-carboxamide hydrochloride;
5-{4-[(4-Methylpiperazin-1-yl)sulfonyl]phenyl}-N-pyridin-3-yl-1H-pyrrolo[2,3-b]pyridine-3-carboxamide hydrochloride;
N-(2-Methoxyethyl)-5-{4-[(4-methylpiperazin-1-yl)sulfonyl]phenyl}-1H-pyrrolo[2,3-b]pyridine-3-carboxamide hydrochloride;
N-(2-Methoxyethyl)-5-[4-[(4-methylpiperazin-1-yl)sulfonyl]-3-(trifluoromethoxy)phenyl]-1H-pyrrolo[2,3-b]pyridine-3-carboxamide hydrochloride;
N-(3-Methoxypropyl)-5-[4-[(4-methylpiperazin-1-yl)sulfonyl]-3-(trifluoromethoxy)phenyl]-1H-pyrrolo[2,3-b]pyridine-3-carboxamide hydrochloride;
5-[4-[(4-Methylpiperazin-1-yl)sulfonyl]-3-(trifluoromethoxy)phenyl]-N-pyridin-3-yl-1H-pyrrolo[2,3-b]pyridine-3-carboxamide hydrochloride;
5-{4-[(4-methylpiperazin-1-yl)sulfonyl]phenyl}-N-[2-(methylsulfonyl)phenyl]-1H-pyrrolo[2,3-b]pyridine-3-carboxamide hydrochloride;
N-(2-Methoxyethyl)-5-[4-(morpholin-4-ylmethyl)phenyl]-1H-pyrrolo[2,3-b]pyridine-3-carboxamide hydrochloride;
N-(3-Methoxypropyl)-5-[4-(piperidin-1-ylmethyl)phenyl]-1H-pyrrolo[2,3-b]pyridine-3-carboxamide hydrochloride;
5-{4-[(4-Methylpiperazin-1-yl)methyl]phenyl}-N-[2-(methylsulfonyl)phenyl]-1H-pyrrolo[2,3-b]pyridine-3-carboxamide hydrochloride;
5-{4-[2-(4-Methylpiperazin-1-yl)ethoxy]phenyl}-N-pyridin-3-yl-1H-pyrrolo[2,3-b]pyridine-3-carboxamide hydrochloride;
N-(3-methoxypropyl)-5-[4-(2-morpholin-4-ylethoxy)phenyl]-1H-pyrrolo[2,3-b]pyridine-3-carboxamide hydrochloride;
N-(3-Methoxypropyl)-5-{4-[2-(4-methylpiperazin-1-yl)ethoxy]phenyl)}-1H-pyrrolo[2,3-b]pyridine-3-carboxamide hydrochloride;
N-(3-Methoxyphenyl)-5-[4-(2-morpholin-4-ylethoxy)phenyl]-1H-pyrrolo[2,3-b]pyridine-3-carboxamide hydrochloride;
N-(2-Methoxyphenyl)-5-[4-(2-morpholin-4-ylethoxy)phenyl]-1H-pyrrolo[2,3-b]pyridine-3-carboxamide hydrochloride; and
5-{4-[2-(4-Methylpiperazin-1-yl)ethoxy]phenyl}-N-(2-morpholin-4-ylethyl)-1H-pyrrolo[2,3-b]pyridine-3-carboxamide hydrochloride;
5-[4-(2-Morpholin-4-ylethoxy)phenyl]-N-pyridin-3-yl-1H-pyrrolo[2,3-b]pyridine-3-carboxamide hydrochloride;
N-[2-(Methylsulfonyl)ethyl]-5-[4-(piperidin-1-ylsulfonyl)phenyl]-1H-pyrrolo[2,3-b]pyridine-3-carboxamide hydrochloride;
5-[4-(Morpholin-4-ylmethyl)phenyl]-N-[2-(2-thienyl)ethyl]-1H-pyrrolo[2,3-b]pyridine-3-carboxamide hydrochloride;
N-Methyl-5-[4-[(4-methylpiperazin-1-yl)sulfonyl]-3-(trifluoromethoxy)phenyl]-1H-pyrrolo[2,3-b]pyridine-3-carboxamide hydrochloride;
N-Methyl-5-{4-[(4-methylpiperazin-1-yl)sulfonyl]phenyl}-1H-pyrrolo[2,3-b]pyridine-3-carboxamide hydrochloride;
N-Methyl-5-[4-(piperidin-1-ylsulfonyl)phenyl]-1H-pyrrolo[2,3-b]pyridine-3-carboxamide hydrochloride;
N-(2-Morpholin-4-ylethyl)-5-(4-morpholin-4-ylphenyl)-1H-pyrrolo[2,3-b]pyridine-3-carboxamide hydrochloride;
N-(2-Morpholin-4-ylethyl)-5-[4-(pyrrolidin-1-ylcarbonyl)phenyl]-1H-pyrrolo[2,3-b]pyridine-3-carboxamide hydrochloride;
5-[4-(Morpholin-4-ylcarbonyl)phenyl]-N-(2-morpholin-4-ylethyl)-1H-pyrrolo[2,3-b]pyridine-3-carboxamide hydrochloride; and
5-[4-(Morpholin-4-ylmethyl)phenyl]-N-pentyl-1H-pyrrolo[2,3-b]pyridine-3-carboxamide hydrochloride.

In yet another aspect of the invention, there is provided compounds being:
Methyl 5-bromo-1H-pyrrolo[2,3-b]pyridine-3-carboxylate;
Methyl 5-{4-[(4-methylpiperazin-1-yl)sulfonyl]phenyl}-1H-pyrrolo[2,3-b]pyridine-3-carboxylate;
Methyl 5-[4-[(4-methylpiperazin-1-yl)sulfonyl]-3-(trifluoromethoxy)phenyl]-1H-pyrrolo[2,3-b]pyridine-3-carboxylate;
Methyl 5-[4-(morpholin-4-ylmethyl)phenyl]-1H-pyrrolo[2,3-b]pyridine-3-carboxylate;
Methyl 5-{4-[(4-methylpiperazin-1-yl)methyl]phenyl}-1H-pyrrolo[2,3-b]pyridine-3-carboxylate;
Methyl 5-[4-(piperidin-1-ylmethyl)phenyl]-1H-pyrrolo[2,3-b]pyridine-3-carboxylate;
Methyl 5-[4-(2-morpholin-4-ylethoxy)phenyl]-1H-pyrrolo[2,3-b]pyridine-3-carboxylate;
Methyl 5-{4-[2-(4-methylpiperazin-1-yl)ethoxy]phenyl}-1H-pyrrolo[2,3-b]pyridine-3-carboxylate;
Methyl 5-[4-(piperidin-1-ylsulfonyl)phenyl]-1H-pyrrolo[2,3-b]pyridine-3-carboxylate;
5-Bromo-N-(2-morpholin-4-ylethyl)-1H-pyrrolo[2,3-b]pyridine-3-carboxamide;
5-Bromo-N-[2-(2-thienyl)ethyl]-1H-pyrrolo[2,3-b]pyridine-3-carboxamide;
5-Bromo-N-methyl-1H-pyrrolo[2,3-b]pyridine-3-carboxamide; and
5-Bromo-N-pentyl-1H-pyrrolo[2,3-b]pyridine-3-carboxamide.

In one embodiment of this aspect of the invention, there is provided compounds being:
Methyl 5-{4-[(4-methylpiperazin-1-yl)sulfonyl]phenyl}-1H-pyrrolo[2,3-b]pyridine-3-carboxylate;
Methyl 5-[4-[(4-methylpiperazin-1-yl)sulfonyl]-3-(trifluoromethoxy)phenyl]-1H-pyrrolo[2,3-b]pyridine-3-carboxylate;
Methyl 5-[4-(morpholin-4-ylmethyl)phenyl]-1H-pyrrolo[2,3-b]pyridine-3-carboxylate;
Methyl 5-{4-[(4-methylpiperazin-1-yl)methyl]phenyl)}-1H-pyrrolo[2,3-b]pyridine-3-carboxylate;
Methyl 5-[4-(piperidin-1-ylmethyl)phenyl]-1H-pyrrolo[2,3-b]pyridine-3-carboxylate;
Methyl 5-[4-(2-morpholin-4-ylethoxy)phenyl]-1H-pyrrolo[2,3-b]pyridine-3-carboxylate;
Methyl 5-{4-[2-(4-methylpiperazin-1-yl)ethoxy]phenyl}-1H-pyrrolo[2,3-b]pyridine-3-carboxylate; and
Methyl 5-[4-(piperidin-1-ylsulfonyl)phenyl]-1H-pyrrolo[2,3-b]pyridine-3-carboxylate.

Listed below are definitions of various terms used in the specification and claims to describe the present invention.

In this specification the term “alkyl” includes both straight and branched chain alkyl groups. The term C₀₋₆alkyl having 0 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl or i-hexyl. The term C₁₋₆alkyl having 1 to 6 carbon atoms and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl. The term C₂₋₆alkyl having 2 to 6 carbon atoms and may be ethyl, n-propyl, i-propyl, n-butyl, i-butyl, s-butyl, t-butyl, n-pentyl, i-pentyl, t-pentyl, neo-pentyl, n-hexyl or i-hexyl.

In the case where a subscript is the integer 0 (zero) the group to which the subscript refers to indicates that the group may be absent, i.e. there is a direct bond between the groups.

The term “cycloalkyl” refers to an optionally substituted, saturated cyclic hydrocarbon ring system. The term “C₃₋₆cycloalkyl” may be, but is not limited to, cyclopropyl, cyclobutyl, cyclopentyl or cyclohexyl.

The term “alkenyl” refers to a straight or branched chain alkenyl group. The term C₂₋₆alkenyl having 2 to 6 carbon atoms and one double bond, and may be, but is not limited to, vinyl, allyl, propenyl, i-propenyl, butenyl, i-butenyl, crotyl, pentenyl, i-pentenyl or hexenyl.

The term “alkynyl” refers to a straight or branched chain alkynyl groups. The term C₂₋₆alkynyl having 2 to 6 carbon atoms and one triple bond, and may be, but is not limited to, ethynyl, propargyl, butynyl, i-butynyl, pentynyl, i-pentynyl or hexynyl.

The term “C₁₋₆alkoxy” may be straight or branched. C₁₋₆alkoxy may be, but is not limited to, methoxy, ethoxy, n-propoxy, i-propoxy, n-butoxy, i-butoxy, s-butoxy, t-butoxy, n-pentyloxy, i-pentyloxy, t-pentyloxy, neo-pentyloxy, n-hexyloxy or i-hexyloxy.

The term “halogen” refers to fluorine, chlorine, bromine and iodine.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl” are phenyl, naphthyl, indanyl or tetralinyl.

The term “heteroaryl” and “5 or 6 membered heteroaromatic ring containing one or more heteroatoms selected from N, O and S” may be, but is not limited to, furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

The term “5- or 6-membered heterocyclic ring containing one or more heteroatoms independently selected from N, O, or S” may optionally contain a carbonyl function and may be, but are not limited to, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S this includes optionally SO and SO₂.

The term “5- or 6-membered saturated, partially saturated or unsaturated ring containing atoms selected from C, N, O or S” may optionally contain a carbonyl function and may be, but are not limited to, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl, thiomorpholinyl, furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl. In the case where the heterocyclic ring contains a heteroatom selected from S this includes optionally SO and SO₂;

It is to be understood that when n is greater than one, R² groups may be the same or different.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have chiral centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

### Methods of Preparations

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis” T. W. Green, P. G. M. Wuts, Wiley-Interscience, New York, 1999.

### Methods of Preparation of Intermediates

The process for the preparation of the intermediates, wherein X, P, Q, R, R¹, R², R³, R⁴, R⁵, R⁶, R⁷, R⁸, R⁹, R¹⁰, R¹¹, R¹², R¹³, R¹⁴, R¹⁵, R¹⁶, A, B, m and n are, unless specified otherwise, defined as in formula I, comprises of:

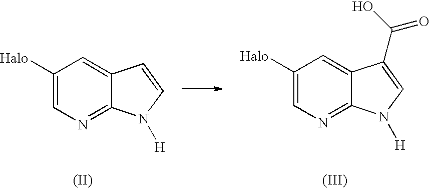

(i) Acylation of a compound of formula II, wherein Halo is halogen e.g. chlorine or bromine, to obtain a compound of formula III, may be carried out by the reaction with a suitable acylating agent such as trichloroacetyl chloride and an appropriate Lewis acid such as aluminum chloride in a suitable solvent or solvent mixtures thereof such as dichloromethane, chloroform, or carbon disulfide, at a temperature range between +20° C. and +60° C. followed by hydrolysis using a suitable base such as aqueous sodium hydroxide, potassium hydroxide, potassium carbonate or sodium hydrogen carbonate in a suitable solvent such as methanol, ethanol, acetone, tetrahydrofuran, diethyl ether, toluene, benzene, dioxane, which is performed in a temperature range between room temperature and +80° C.

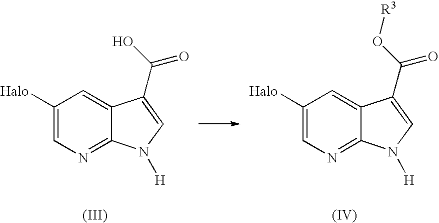

(ii) Estrification of a compound of formula III wherein Halo is halogen e.g. chlorine or bromine to obtain a compound of formula IV wherein R³ is defined as above may be carried out,
a) via transesterfication using the appropriate ester. The ester may be formed from e.g. an appropriate acyl halide such as acetyl chloride or acetyl bromide and an appropriate alcohol such as methanol or ethanol. The reaction can be performed in a temperature range between room temperature and +80° C.
b) with an appropriate alcohol such as methanol or ethanol and a catalyst such as sulfuric acid or p-toluenesulfonic acid and the reaction may be performed in a temperature range between room temperature and +80° C. Either using the alcohol as the solvent or by removal of the formed water to drive the reaction to completion.
c) via alcoholysis of a formed acyl halide by treatment of the compound III with a suitable inorganic acid halide such as thionyl chloride, phosphorous pentachloride, phosphorous trichloride, phosphorous oxychloride or by treatment with oxalyl chloride (with or without a catalytic amount of N,N-dimethylformamide) and subsequent treatment with an appropriate alcohol such as methanol or ethanol. The reaction can be performed in a temperature range between 0° C. and +110° C.

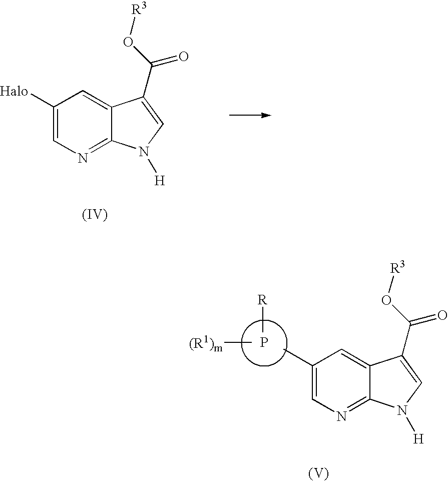

(iii) De-halogen coupling of a compound of formula IV, wherein Halo is halogen and R³ is as defined above, with a suitable aryl species to give a compound of formula V, may be carried out by,
a) reaction with an appropriate aryl boronic acid or an aryl boronic ester. The reaction may be carried out using a suitable palladium catalyst such as Pd(PPh₃)₄, [1,1′bis(diphenylphosphino)ferrocene]palladium(II) chloride dichloromethane, Pd(OAc)₂ or Pd₂(dba)₃ together with a suitable ligand such as P(tert-butyl)₃ or 2-(dicyclohexylphosphino)biphenyl. A suitable base such as an alkyl amine e.g. triethylamine, or potassium carbonate, sodium carbonate, cesium carbonate, sodium hydroxide or cesium fluoride may be used in the reaction, which is performed in a temperature range between +20° C. and +160° C. using an oil bath or a microwave oven in a suitable solvent or solvent mixture such as toluene, tetrahydrofuran, dimethoxyethane/water, N,N-dimethylformamide or dioxane.

The boronic acid or boronic ester can be formed in situ by reaction of the corresponding arylhalide such as an arylbromide with an alkyllithium reagent such as butyllithium and a suitable boron compound e.g. trimethyl borat, tributyl borate or triisopropyl borate. The reaction may be performed in an aprotic solvent e.g. tetrahydrofuran or hexane in a temperature range between −78° C. and +20° C.;

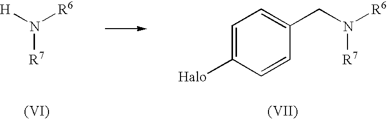

(iv) alkylation of a compound of formula VI, to obtain a compound of formula VII, wherein Halo is halogen such as bromine or chlorine, may be carried out by reaction of a compound of formula VI with a suitable alkylhalide such as an alkylbromid, chloride or iodide in a suitable solvent such as methanol, ethanol, dichloromethane, chloroform, N,N-dimethylamide or acetonitril with or without a suitable base e.g. a trialkylamine base such as triethylamine, or a other appropriate base such as sodium hydroxide, potassium hydroxide or potassium carbonate and at a reaction temperature between +20° C. and reflux.

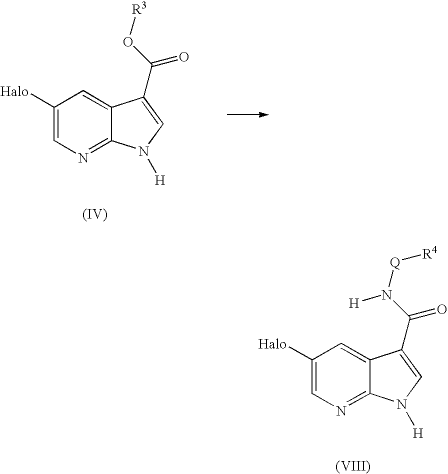

(v) amidation of a compound of formula IV, to obtain a compound of formula VIII, may be carried out by treating a compound of formula IV with the appropriate amine, R⁴QNH₂. The reaction may be performed neat or using a suitable solvent such as N,N-dimethylformamide, toluene, dichloromethane, chloroform or ethyl acetate at a temperature ranging from −25° C. to +150° C. The reaction may be aided by using a base such as potassium carbonate, triethylamine or 1,8-diazabicyclo[5.4.0]undec-7-ene or an acid such as trimethylaluminum or p-toulenesulfonic acid.

### Methods of Preparation of End Products

Another objective of the invention are processes for the preparation of a compound of general formula I, wherein X, P, Q, R, R¹, R², R³, R⁴, R⁵, R⁶, R⁷, R⁸, R⁹, R¹⁰, R¹¹, R¹², R¹³, R¹⁴, R¹⁵ R¹⁶, A, B, m and n are, unless specified otherwise, defined as in formula I, comprises of:

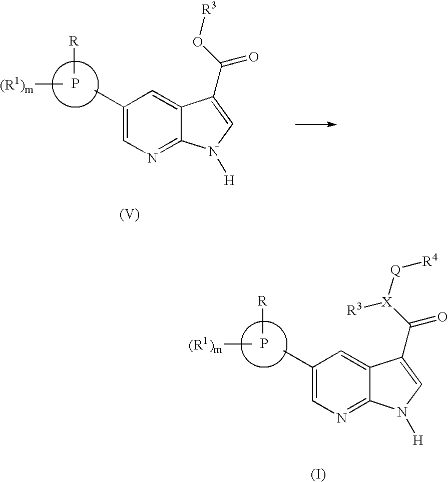

### A

amidation of a compound of formula V, to obtain a compound of formula I, may be carried out by treating a compound of formula V with the appropriate amine, HN(R³)QR⁴. The reaction may be performed neat or using a suitable solvent mixture or solvents selected from solvents such as benzene, toluene, N,N-dimethylformamide, dichloromethane or ethyl acetate at a temperature ranging from −25° C. to +150° C. The reaction may be aided by using a base such as potassium carbonate, triethylamine, diisopropylethylamine or 1,8-diazabicyclo[5.4.0]undec-7-ene or an acid such as trimethylaluminum or p-toulenesulfonic acid.

Consequently, in one aspect of the present invention, there is provided a process for preparing a compound of formula I, wherein X, P, Q, R, R¹, R², R³, R⁴, R⁵, R⁶, R⁷, R⁸, R⁹, R¹⁰, R¹¹, R¹², R¹³, R¹⁴, R¹⁵ R¹⁶, A, m and n are, unless specified otherwise, defined as in formula I, comprising:

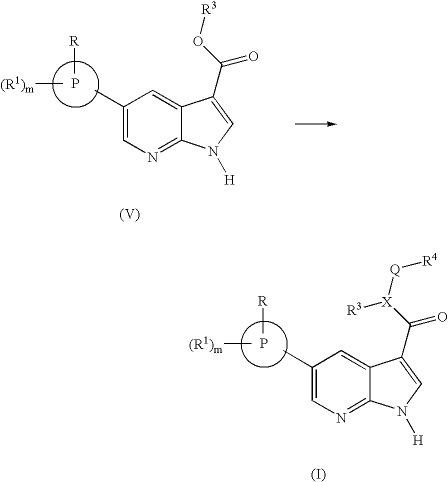

amidation of a compound of formula V, to obtain a compound of formula I, by treating a compound of formula V with an amine, HN(R³)QR⁴ preformed neat or by using a suitable solvent, optionally with the addition of a base or an acid.

### B

de-halogen coupling, wherein R⁴ and Q are substituents that are not susceptible to certain agents in the reaction, of a compound of formula VIII with a suitable aryl species to give a compound of formula I:

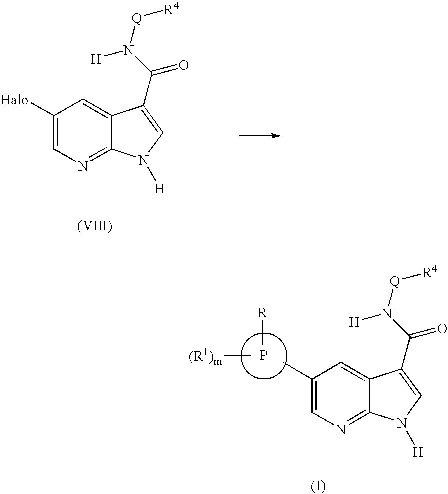

Thus, the de-halogen coupling according to process B may be carried out by coupling of a compound of formula VIII with:

an appropriate aryl boronic acid or a bornic ester. The reaction may be carried out using a suitable palladium catalyst such as Pd(PPh₃)₄, Pd(OAc)₂ or [1,1′bis(diphenylphosphino)ferrocene]palladium(II) chloride dichloromethane, with or without a suitable ligand such as P(tert-butyl)₃ or 2-(dicyclohexylphosphino)biphenyl. A suitable base such as an alkyl amine e.g. triethyl amine, or potassium carbonate, sodium carbonate, sodium hydroxide or cesium fluoride may be used in the reaction, which is performed in the temperature range between +20° C. and +160° C. using an oil bath or in a microwave oven in a suitable solvent or solvent mixture such as toluene, tetrahydrofuran, dimethoxyethane/water or N,N-dimethylformamide.

The boronic acid or boronic ester can be formed in situ by reaction of the corresponding arylhalide such as an arylbromide, e.g. compound of formula VII, with an alkyllithium reagent such as butyllithium and a suitable boron compound e.g. trimethyl borat, tributyl borate or triisopropyl borate. The reaction may be performed in an aprotic solvent e.g. tetrahydrofuran or hexane in a temperature range between −78° C. and +20° C.;

Consequently, in one aspect of the present invention, there is provided a process for preparing a compound of formula I, wherein X, P, Q, R, R¹, R², R³, R⁴, R⁵, R⁶, R⁷, R⁸, R⁹, R¹⁰, R¹¹, R¹², R¹³, R¹⁴, R¹⁵ R¹⁶, A, m and n are, unless specified otherwise, defined as in formula I, comprising:

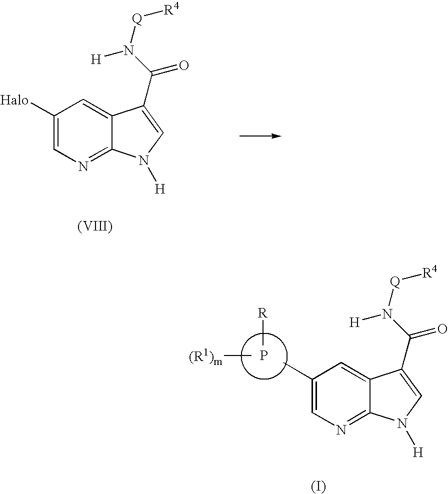

de-halogen coupling of a compound of formula VIII with an appropriate aryl species in a suitable solvent to give a compound of formula I.

The hydrochloric salt of a compound of formula I may be obtained from a compound of formula I by treatment with hydrochloric acid at a temperature range between 0° C. and +25° C., in a suitable solvent such as dichloromethane, tetrahydrofuran or dichloromethane/methanol mixture.

### WORKING EXAMPLES

Below follows a number of non-limiting examples of compounds of the invention. Starting materials are commercially available or known in the literature.

### Example 1

### Methyl 5-bromo-1H-pyrrolo[2,3-b]pyridine-3-carboxylate

A solution of 5-bromo-1H-pyrrolo[2,3-b]pyridine (0.200 g, 1.01 mmol; described in: Mazeas, D. et al, Heterocycles 1999, 50, 1065-1080) in dichloromethane (12 mL) was added to a suspension of aluminum chloride (0.704 g, 5.28 mmol) in dichloromethane (5 mL) under an atmosphere of nitrogen. The resulting mixture was stirred at room temperature for 40 min to give a brownish solution. Trichloroacetyl chloride (0.56 mL, 5.0 mmol) was added and the mixture was stirred at room temperature for 17 h. Methanol (10 mL) was added and the solvent was evaporated in vacuo. The residue was treated with aqueous potassium hydroxide (3 M, 10 mL) and methanol (5 mL) and heated at 60° C. for 1 h and 15 min. The mixture was allowed to cool to room temperature and the pH was adjusted to 1-2 using aqueous hydrochloric acid (2 M). The aqueous phase was extracted with ethyl acetate, dried over sodium sulfate, and the solvent was evaporated to give a brown residue. Acetyl chloride (10 mL) was added dropwise to cooled methanol (0° C., 20 mL). The resulting solution was added to a solution of the brown residue in methanol (10 mL) at room temperature, and the resulting mixture was heated at reflux for 3 h. The mixture was allowed to cool to room temperature and the solvent was evaporated to give a yellow solid. The crude product was purified on a silica gel column using a gradient, ethyl acetate/heptane mixture (10, 20, 30, 40, 50% ethyl acetate), as the eluent to give 0.165 g (64% yield) of the title compound as a pale pink solid: ¹H NMR (DMSO-d₆, 300 MHz) δ 12.80 (br s, 1H), 8.41 (s, 2H), 8.30 (d, J=3.0 Hz, 1H), 3.83 (s, 3H); ¹³C NMR (DMSO-d₆, 75 MHz) δ 163.9, 147.0, 144.1, 134.5, 130.5, 119.6, 113.1, 105.0, 51.1; MS (ES) m/z 255 and 257 (M⁺+1).

### Example 2

### Methyl 5-{4-[(4-methylpiperazin-1-yl)sulfonyl]phenyl}-1H-pyrrolo[2,3-b]pyridine-3-carboxylate

n-Butyllithium (4 mL, 1.6 M in hexane, 6.4 mmol) was added dropwise over 15 min to a cooled (−70° C.) solution of 1-[(4-bromophenyl)sulfonyl]-4-methylpiperazine (0.651 g, 2.04 mmol) and tributyl borate (1.65 mL, 6.14 mmol) in tetrahydrofuran (20 mL) under an atmosphere of nitrogen and the resulting mixture was stirred at −70° C. for 1 h. The mixture was allowed to warm to room temperature and aqueous hydrochloric acid (3.5 mL, 3 M, 10.5 mmol) was added followed by the addition of sodium carbonate (2.16 g, 20.4 mmol). A suspension of methyl 5-bromo-1H-pyrrolo[2,3-b]pyridine-3-carboxylate (0.372 g, 1.46 mmol) in tetrahydrofuran (20 mL) was added followed by addition of [1,1′-bis(diphenylphosphino)ferrocene]palladium(II) chloride dichloromethane adduct (0.166 g, 0.20 mmol), and the resulting mixture was heated at 65° C. for 21 h. The mixture was allowed to cool to room temperature and the solvent was evaporated in vacuo. The residue was diluted with dichloromethane/methanol, (1:1, 200 mL), silica (5 g) was added and the solvent was evaporated. Purification on a silica gel column, using a gradient dichloromethane/methanol mixture (1, 2, 3, 4, 5, 6, 8% methanol, 250 mL/concentration) as the eluent, gave 0.52 g (86% yield) of the title compound as an orange solid: ¹H NMR (DMSO-d₆, 300 MHz) δ 12.72 (br s, 1H), 8.71 (d, J=2.1 Hz, 1H), 8.58 (d, J=1.8 Hz, 1H), 8.32 (s, 1H), 8.02 (d, J=8.1 Hz, 2H), 7.84 (d, J=7.8 Hz, 2H), 3.86 (s, 3H), 2.94 (m, 4H), 2.38 (m, 4H), 2.14 (s, 3H); MS (ES) m/z 415 (M⁺+1).

The Examples 34 was synthesized as described for Example 2.

### Example 3

### Methyl 5-[4-[(4-methylpiperazin-1-yl)sulfonyl]-3-(trifluoromethoxy)phenyl]-1H-pyrrolo[2,3-b]pyridine-3-carboxylate

Starting materials: 1-{[4-bromo-2-(trifluoromethoxy)phenyl]sulfonyl}-4-methylpiperazine and methyl 5-bromo-1H-pyrrolo[2,3-b]pyridine-3-carboxylate, yield 70%: ¹H NMR (DMSO-d₆, 300 MHz) δ 12.77 (br s, 1H), 8.73 (d, J=2.1 Hz, 1H), 8.59 (d, J=2.1 Hz, 1H), 8.34 (s, 1H), 8.01 (m, 2H), 7.89 (s, 1H), 3.85 (s, 3H), 3.12 (m, 4H), 2.37 (m, 4H), 2.17 (s, 3H); MS (ES) m/z 499 (M⁺+1).

### Example 4

### Methyl 5-[4-(morpholin-4-ylmethyl)phenyl]-1H-pyrrolo[2,3-b]pyridine-3-carboxylate

Starting materials: 4-(4-bromobenzyl)morpholine and methyl 5-bromo-1H-pyrrolo[2,3-b]pyridine-3-carboxylate, yield 37%: ¹H NMR (DMSO-d₆, 300 MHz) δ 12.61 (s, 1H), 8.61 (d, J=2.1 Hz, 1H), 8.47 (d, J=1.8 Hz, 1H), 8.27 (d, J=2.4 Hz, 1H), 7.68 (d, J=8.1 Hz, 2H), 7.44 (d, J=8.1 Hz, 2H), 3.85 (s, 3H), 3.59 (m, 4H), 3.52 (s, 2H), 2.39 (m, 4H).

### Example 5

### Methyl 5-{4-[(4-methylpiperazin-1-yl)methyl]phenyl}-1H-pyrrolo[2,3-b]pyridine-3-carboxylate

t-Butyllithium (2.4 mL, 1.7 M in hexane, 4.08 mmol) was added dropwise over 15 min to a cooled (−70° C.) solution of 1-(4-bromobenzyl)-4-methylpiperazine (0.500 g, 1.86 mmol; described in: Organ M. G. et al J. Comb. Chem. 2001, 3, 473) in anhydrous tetrahydrofuran (10 mL) followed by addition of tributyl borate (1.2 mL, 4.47 mmol) over 5 min under an atmosphere of nitrogen. The resulting yellow solution was stirred at −70° C. for 1 h and then allowed to warm to room temperature. Hydrochloric acid (3 mL, 3 M aq) was added followed by addition of sodium carbonate (2.0 g, 18.9 mmol). After stirring for 10 min a suspension of methyl 5-bromo-1H-pyrrolo[2,3-b]pyridine-3-carboxylate (0.331 g, 1.30 mmol) in tetrahydrofuran (15 mL) was added followed by addition of [1,1′-bis(diphenylphosphino)ferrocene]palladium(II) chloride dichloromethane adduct (0.147 g, 0.18 mmol), and the resulting mixture was stirred at 65° C. for 17 h. The mixture was allowed to cool to room temperature and the pH was adjusted to 9 with aqueous potassium hydroxide (1.2 mL, 3 M), and additional [1,1′-bis(diphenylphosphino)ferrocene]palladium(II) chloride dichloromethane adduct (0.147 g, 0.18 mmol) was added and the mixture was heated at 65° C. for 2 days. The solvents were evaporated in vacuo. Methanol, dichloromethane, and silica were added and the solvents evaporated. The resulting residue was purified on a silica gel column using a gradient, methanol/dichloromethane mixture (2, 5, 7, 10% methanol and 1% NH₃ (aq)), as the eluent. The title compound was isolated, washed with ethyl acetate, filtrated, and dried in vacuo to give 0.051 g (11% yield): ¹H NMR (DMSO-d₆, 300 MHz) δ 12.59 (s, 1H), 8.61 (d, J=1.8 Hz, 1H), 8.47 (d, J=2.1 Hz, 1H), 8.27 (s, 1H), 7.67 (d, J=8.1 Hz, 2H), 7.42 (d, J=8.1 Hz, 2H), 3.85 (s, 3H), 3.51 (s, 2H), 2.39 (m, 4H), 2.33 (m, 4H), 2.15 (s, 3H); MS (ES) m/z 365 (M⁺+1).

### Example 6

### Methyl 5-[4-(piperidin-1-ylmethyl)phenyl]-1H-pyrrolo[2,3-b]pyridine-3-carboxylate

The title compound was synthesized as described for Example 5 using 1-(4-bromobenzyl)piperidine and methyl 5-bromo-1H-pyrrolo[2,3-b]pyridine-3-carboxylate, yield 11%. Purification by preparative HPLC (column: Xterra C8; eluent: 0.05 M NH₄Ac(aq) in acetonitrile (20 to 60% acetonitrile)): ¹H NMR (DMSO-d₆, 300 MHz) δ 8.61 (d, J=2.4 Hz, 1H), 8.47 (d, J=1.8 Hz, 1H), 8.27 (s, 1H), 7.66 (d, J=8.1 Hz, 2H), 7.41 (d, J=8.1 Hz, 2H), 3.85 (s, 3H), 3.47 (s, 2H), 2.35 (m, 4H), 1.50 (m, 4H), 1.41 (m, 2H); MS (ES) m/z 350 (M⁺+1).

### Example 7

### Methyl 5-[4-(2-morpholin-4-ylethoxy)phenyl]-1H-pyrrolo[2,3-b]pyridine-3-carboxylate

Triisopropyl borate (0.39 mL, 1.7 mmol) was added to a stirred solution of 4-[2-(4-bromophenoxy)ethyl]morpholine (0.163 g, 0.57 mmol; described in: Lednicer, D., et al, J. Med. Chem. 1965, 8, 52-57) in anhydrous tetrahydrofuran (4 mL). The resulting solution was cooled to −70° C. and n-butyllithium (1.1 mL, 1.6 M in hexane, 1.76 mmol) was added dropwise. The mixture was stirred at −70° C. for 1 h and was then allowed to warm to room temperature. Hydrochloric acid (aq. 2.85 mL, 1 M, 2.85 mmol) was added and the mixture was stirred for 2 min, followed by addition of sodium carbonate (0.604 g, 5.7 mmol). After stirring for another 2 min, methyl 5-bromo-1H-pyrrolo[2,3-b]pyridine-3-carboxylate (0.100 g, 0.4 mmol) and [1,1′-bis(diphenylphosphino)ferrocene]palladium(II) chloride dichloromethane adduct (0.047 g, 0.057 mmol) were added and the resulting mixture was heated at 65° C. over night. The solvent was evaporated and the residue was purified on a silica gel column using dichloromethane/methanol, (95:5), as the eluent. Re-crystallization from acetonitrile gave 0.048 g (31% yield) of the title compound as crystals:

¹H NMR (CDCl₃, 400 MHz) δ 10.06 (m, 1H), 8.57 (m, 2H), 8.06 (s, 1H), 7.58 (m, 2H), 7.02 (m, 2H), 4.29 (m, 2H), 3.94 (s, 3H), 3.85 (m, 4H), 2.99 (m, 2H), 2.77 (m, 4H).

The Examples 8-9 were synthesized as described for Example 7.

### Example 8

### Methyl 5-{4-[2-(4-methylpiperazin-1-yl)ethoxy]phenyl}-1H-pyrrolo[2,3-b]pyridine-3-carboxylate

Starting materials: 1-[2-(4-bromophenoxy)ethyl]-4-methylpiperazine (described in: Ide, et al, J. Am. Chem. Soc., 1954, 76, 1122-1125) and methyl 5-bromo-1H-pyrrolo[2,3-b]pyridine-3-carboxylate, yield 56%: ¹H NMR (CDCl₃, 400 MHz) δ 10.61 (br s, 1H), 8.58 (m, 2H), 8.08 (s, 1H), 7.57 (m, 2H), 7.02 (m, 2H), 4.17 (m, 2H), 3.94 (s, 3H), 2.87 (m, 2H), 2.70 (m, 4H), 2.56 (m, 4H), 2.34 (s, 3H); MS (ES) m/z 395 (M⁺+1).

### Example 9

### Methyl 5-[4-(piperidin-1-ylsulfonyl)phenyl]-1H-pyrrolo[2,3-b]pyridine-3-carboxylate

Starting materials: 1-[(4-bromophenyl)sulfonyl]piperidine and methyl 5-bromo-1H-pyrrolo[2,3-b]pyridine-3-carboxylate, yield 81%: ¹H NMR (CDCl₃, 400 MHz) δ 8.71 (s, 1H), 8.63 (m, 1H), 8.09 (s, 1H), 7.88 (d J=8.3 Hz, 2H), 7.80 (d, J=8.3 Hz, 2H), 3.96 (s, 3H), 3.06 (m, 4H), 1.67 (m, 4H), 1.45 (m, 3H); MS (TSP) m/z 400 (M⁺+1).

### Example 10

### N-(3-Methoxypropyl)-5-{4-[(4-methylpiperazin-1-yl)sulfonyl]phenyl}-1H-pyrrolo[2,3-b]pyridine-3-carboxamide hydrochloride

3-Methoxypropylamine (0.021 g, 0.24 mmol) was added to a cooled (0° C.) solution of methyl 5-{4-[(4-methylpiperazin-1-yl)sulfonyl]phenyl}-1H-pyrrolo[2,3-b]pyridine-3-carboxylate (0.047 g, 0.11 mmol) in benzene (5 mL) followed by addition of trimethylaluminum (0.3 mL, 2.0 M in toluene, 0.6 mmol), under an atmosphere of nitrogen. The resulting mixture was refluxed for 2 h. The mixture was allowed to cool down to room temperature, saturated aqueous sodium hydrogen carbonate (10 mL) was added, and the resulting mixture was stirred for 30 min. The mixture was diluted with water (10 mL). The aqueous phase was extracted with a mixture of chloroform/methanol (95:5, 3×10 mL), dried over sodium sulfate and the solvent was evaporated. The resulting residue was purified by preparative HPLC (column: Xterra C8; eluent: 0.05 M NH₄Ac(aq) in acetonitrile (10 to 60% acetonitrile)) to give 0.025 g of the base. The base was dissolved in chloroform (1 mL) and methanol (0.5 mL) and hydrochloric acid (1 M in diethyl ether, 0.1 mL) was added followed by addition of diethyl ether until precipitation. The solid was collected by filtration and dried in vacuo to give 25 mg (43% yield) of the title compound as a pale yellow solid: ¹H NMR (DMSO-d₆, 300 MHz) δ 12.30 (s, 1H), 10.57 (m, 1H), 8.77 (d, J=2.4 Hz, 1H), 8.68 (d, J=1.8 Hz, 1H), 8.24 (d, J=3.0 Hz, 1H), 8.14 (m, 1H), 8.06 (d, J=8.4 Hz, 2H), 7.89 (d, J=8.4 Hz, 2H), 3.83 (m, 2H), 3.47 (m, 2H), 3.40 (m, 4H), 3.32 (m, 2H), 3.25 (s, 3H), 2.75 (m, 5H), 1.78 (m, 2H); MS (ES) m/z 472 (M⁺+1).

The Examples 11-18 were synthesized as described for Example 10.

### Example 11

### 5-{4-[(4-Methylpiperazin-1-yl)sulfonyl]phenyl}-N-pyridin-3-yl-1H-pyrrolo[2,3-b]pyridine-3-carboxamide hydrochloride

Starting materials: 3-aminopyridine and methyl 5-{4-[(4-methylpiperazin-1-yl)sulfonyl]phenyl}-1H-pyrrolo[2,3-b]pyridine-3-carboxylate, yield 38%:

After extraction with chloroform, a large amount of the product was still in the aqueous phase that was freeze-dried. The combined residues were purified on a silica gel column using a gradient of dichloromethane/methanol (2-14% methanol) as the eluent: ¹H NMR (DMSO-d₆, 300 MHz) δ 12.73 (m, 1H), 11.28 (s, 1H), 10.67 (m, 1H), 9.52 (m, 1H), 8.96 (d, J=2.7 Hz, 1H), 8.88 (d, J=9.3 Hz, 1H), 8.83 (d, J=2.1 Hz, 1H), 8.77 (d, J=2.4 Hz, 1H), 8.60 (d, J=5.4 Hz, 1H), 8.09 (d, J=8.5 Hz, 2H), 8.01 (dd, J=8.6, 5.6 Hz, 1H), 7.91 (d, J=8.5 Hz, 2H), 3.84 (m, 2H), 3.47 (m, 2H), 3.17 (m, 2H), 2.75 (s, 3H), 2.74 (m, 2H); MS (ES) m/z 477 (M⁺+1).

### Example 12

### N-(2-Methoxyethyl)-5-{4-[(4-methylpiperazin-1-yl)sulfonyl]phenyl}-1H-pyrrolo[2,3-b]pyridine-3-carboxamide hydrochloride

Starting materials: 2-methoxyethylamine and methyl 5-{4-[(4-methylpiperazin-1-yl)sulfonyl]phenyl}-1H-pyrrolo[2,3-b]pyridine-3-carboxylate, yield 58%: ¹H NMR (DMSO-d₆, 300 MHz) δ 12.37 (s, 1H), 11.21 (m, 1H), 8.79 (d, J=1.8 Hz, 1H), 8.69 (d, J=2.1 Hz, 1H), 8.31 (m, 2H), 8.05 (d, J=8.1 Hz, 2H), 7.89 (d, J=8.4 Hz, 2H), 3.82 (m, 2H), 3.46 (m, 6H), 3.28 (s, 3H), 3.16 (m, 2H), 2.82 (m, 2H), 2.73 (m, 3H); MS (ES) m/z 458 (M⁺+1).

### Example 13

### N-(2-Methoxyethyl)-5-[4-[(4-methylpiperazin-1-yl)sulfonyl]-3-(trifluoromethoxy)phenyl]-1H-pyrrolo[2,3-b]pyridine-3-carboxamide hydrochloride

Starting materials: 2-methoxyethylamine and methyl 5-[4-[(4-methylpiperazin-1-yl)sulfonyl]-3-(trifluoromethoxy)phenyl]-1H-pyrrolo[2,3-b]pyridine-3-carboxylate. Heating was continued at reflux for 5 h, yield 58%: ¹H NMR (DMSO-d₆, 300 MHz) δ 12.37 (s, 1H), 10.67 (m, 1H), 8.74 (m, 2H), 8.29 (m, 1H), 8.23 (m, 1H), 8.05 (m, 2H), 7.93 (s, 1H), 3.85 (m, 2H), 3.47 (m, 6H), 3.28 (s, 3H), 3.11 (m, 4H), 2.78 (m, 3H); MS (ES) m/z 542 (M⁺+1).

### Example 14

### N-(3-Methoxypropyl)-5-[4-[(4-methylpiperazin-1-yl)sulfonyl]-3-(trifluoromethoxy)phenyl]-1H-pyrrolo[2,3-b]pyridine-3-carboxamide hydrochloride

Starting materials: 3-methoxypropylamine and methyl 5-[4-[(4-methylpiperazin-1-yl)sulfonyl]-3-(trifluoromethoxy)phenyl]-1H-pyrrolo[2,3-b]pyridine-3-carboxylate. Heating was continued at reflux for 6 h. The solvents were evaporated in vacuo and the resulting residue was purified on a silica gel column using a gradient of dichloromethane/methanol (1 to 10% methanol) as the eluent, yield 51%: ¹H NMR (DMSO-d₆, 300 MHz) δ 12.37 (s, 1H), 10.65 (m, 1H), 8.73 (m, 2H), 8.25 (m, 1H), 8.15 (m, 1H), 8.05 (m, 2H), 7.93 (s, 1H), 3.86 (m, 2H), 3.50 (m, 2H), 3.40 (m, 2H), 3.32 (m, 2H), 3.25 (s, 3H), 3.09 (m, 4H), 2.79 (br s, 3H), 1.78 (quint, J=6.6 Hz, 2H); MS (ES) m/z 556 (M⁺+1).

### Example 15

### 5-[4-[(4-Methylpiperazin-1-yl)sulfonyl]-3-(trifluoromethoxy)phenyl]-N-pyridin-3-yl-1H-pyrrolo[2,3-b]pyridine-3-carboxamide hydrochloride

Starting materials: 3-aminopyridine and methyl 5-[4-[(4-methylpiperazin-1-yl)sulfonyl]-3-(trifluoromethoxy)phenyl]-1H-pyrrolo[2,3-b]pyridine-3-carboxylate, yield 53%. Heating was continued at reflux for 5 h. The solvents were evaporated in vacuo and the resulting residue was purified on a silica gel column using a gradient of dichloromethane/methanol (1 to 12% methanol) as the eluent, yield 53%: ¹H NMR (DMSO-d₆, 300 MHz) δ 12.79 (m, 1H), 11.26 (s, 1H), 10.97 (m, 1H), 9.50 (m, 1H), 8.97 (d, J=2.4 Hz, 1H), 8.83 (m, 3H), 8.59 (d, J=5.1 Hz, 1H), 8.02 (m, 4H), 3.87 (m, 2H), 3.48 (m, 2H), 3.12 (m, 4H), 2.78 (s, 3H); MS (ES) m/z 561 (M⁺+1).

### Example 16

### 5-{4-[(4-Methylpiperazin-1-yl)sulfonyl]phenyl}-N-[2-(methylsulfonyl)phenyl]-1H-pyrrolo[2,3-b]pyridine-3-carboxamide hydrochloride

Starting materials: 2-(methylsulfonyl)phenylamine hydrochloride and methyl 5-{4-[(4-methylpiperazin-1-yl)sulfonyl]phenyl}-1H-pyrrolo[2,3-b]pyridine-3-carboxylate. Heating was continued at reflux for 5 h. The solvents were evaporated in vacuo and the resulting residue was purified on a silica gel column using a gradient of dichloromethane/methanol (1 to 10% methanol) as the eluent, yield 16%: ¹H NMR (DMSO-d₆, 300 MHz) δ 12.72 (m, 1H), 10.14 (s, 1H), 10.04 (m, 1H), 8.79 (m, 2H), 8.34 (d, J=8.1 Hz, 1H), 8.29 (d, J=2.7 Hz, 1H), 8.11 (d, J=8.4 Hz, 2H), 7.96 (m, 1H), 7.91 (d, J=8.4 Hz, 2H), 7.78 (m, 1H), 7.43 (m, 1H), 3.84 (m, 2H), 3.48 (m, 2H), 3.33 (s, 3H), 2.77 (m, 3H), 2.67 (m, 2H); MS (ES) m/z 554 (M⁺+1).

### Example 17

### N-(2-Methoxyethyl)-5-[4-(morpholin-4-ylmethyl)phenyl]-1H-pyrrolo[2,3-b]pyridine-3-carboxamide hydrochloride

Starting material: 2-methoxyethylamine and methyl 5-[4-(morpholin-4-ylmethyl)phenyl]-1H-pyrrolo[2,3-b]pyridine-3-carboxylate. The solvent was evaporated and the residue was purified on a silica gel column using a gradient of dichloromethane/methanol (2 to 10% methanol) as the eluent, yield 47%: ¹H NMR (DMSO-d₆, 300 MHz) δ 12.22 (s, 1H), 10.82 (m, 1H), 8.71 (d, J=1.8 Hz, 1H), 8.61 (d, J=2.4 Hz, 1H), 8.24 (d, J=3.0 Hz, 1H), 8.17 (m, 1H), 7.83 (d, J=8.1 Hz, 2H), 7.70 (d, J=8.1 Hz, 2H), 4.40 (m, 2H), 3.94 (m, 4H), 3.76 (m, 2H), 3.46 (m, 4H), 3.29 (s, 3H), 3.14 (m, 2H).

### Example 18

### N-(3-Methoxypropyl)-5-[4-(piperidin-1-ylmethyl)phenyl]-1H-pyrrolo[2,3-b]pyridine-3-carboxamide hydrochloride

Starting materials: 3-methoxypropylamine and methyl 5-[4-(piperidin-1-ylmethyl)phenyl]-1H-pyrrolo[2,3-b]pyridine-3-carboxylate, yield 21%: MS (ES) m/z 407 (M⁺+1).

### Example 19

### 5-{4-[(4-Methylpiperazin-1-yl)methyl]phenyl}-N-[2-(methylsulfonyl)phenyl]-1H-pyrrolo[2,3-b]pyridine-3-carboxamide hydrochloride

2-(Methylsulfonyl)phenylamine hydrochloride (0.103 g, 0.50 mmol) and diisopropylethylamine (0.17 mL, 0.98 mmol) were added to a stirred suspension of methyl 5-{4-[(4-methylpiperazin-1-yl)methyl]phenyl}-1H-pyrrolo[2,3-b]pyridine-3-carboxylate (0.060 g, 0.16 mmol) in benzene (5 mL) under an atmosphere of nitrogen and the resulting mixture was stirred at room temperature for 15 min. The mixture was cooled (0° C.), trimethylaluminum (0.8 mL, 2.0 M in toluene, 1.6 mmol) was added, and the yellow solution was heated at reflux for 5 h. The mixture was cooled to room temperature, saturated aqueous sodium hydrogen carbonate (5 mL) was added, and the resulting mixture was stirred for 1 h and 30 min. The mixture was diluted with water (5 mL) and concentrated in vacuo. The residue was purified on a silica gel column using a gradient methanol/dichloromethane mixture (2 to 12% methanol and 1% NH₃ (aq)) as the eluent followed by purification by preparative HPLC (column: Xterra C8; eluent: 0.05 M NH₄Ac(aq) in acetonitrile (20 to 60% acetonitrile)) to give 0.025 g of the base. The base was dissolved in chloroform (1.5 mL) and methanol (0.5 mL) and hydrochloric acid (1 M in diethyl ether, 0.1 mL) was added followed by addition of diethyl ether until precipitation. The solid was collected by filtration and dried at 85° C. in vacuo to give 0.026 g (28% yield) of the title compound: ¹H NMR (DMSO-d₆, 300 MHz) δ 12.62 (s, 1H), 10.12 (s, 1H), 8.69 (d, J=3.0 Hz, 2H), 8.36 (d, J=8.1 Hz, 1H), 8.25 (d, J=2.4 Hz, 1H), 7.96 (d, J=7.5 Hz, 1H), 7.78 (m, 3H), 7.58 (m, 2H), 7.42 (m, 1H), 3.45 (m, 8H), 3.14 (m, 4H), 2.78 (m, 4H); MS (ES) m/z 504 (M⁺+1).

### Example 20

### 5-{4-[2-(4-Methylpiperazin-1-yl)ethoxy]phenyl}-N-pyridin-3-yl-1H-pyrrolo[2,3-b]pyridine-3-carboxamide hydrochloride

3-Aminopyridine (0.018 g, 0.19 mmol) was added to a stirred solution of methyl 5-{4-[2-(4-methylpiperazin-1-yl)ethoxy]phenyl}-1H-pyrrolo[2,3-b]pyridine-3-carboxylate (0.025 g, 0.06 mmol) in toluene (4 mL) under an atmosphere of argon. The resulting mixture was cooled at 0° C. and trimethylaluminum (0.3 mL, 2 M in toluene, 0.6 mmol) was added dropwise. The mixture was stirred at 0° C. for 5 min, the cooling was removed, and the reaction mixture was heated at 110° C. for 2 h. The reaction mixture was allowed to cool to room temperature, and a saturated aqueous solution of sodium hydrogen carbonate (2 mL) was added. The resulting mixture was stirred for 30 min followed by dilution with water (2 mL), and the stirring was continued for another 40 min. The mixture was centrifuged, the solvents were removed, the resulting residue was washed with toluene, the mixture centrifuged, and the toluene removed. The resulting solid was treated with methanol and the formed suspension was stirred for 45 min, centrifuged, and the methanol was removed. Chloroform was added and un-dissolved material was removed by filtration. The solution was treated with hydrochloric acid (4 M, in diethyl ether), the formed precipitate was removed by filtration, and dried in vacuo at 60° C. to give 6 mg (20% yield) of the title compound as a solid: MS (ES) m/z 457 (M⁺+1).

The Examples 21-25 was synthesized as described for Example 20.

### Example 21

### N-(3-Methoxypropyl)-5-[4-(2-morpholin-4-ylethoxy)phenyl]-1H-pyrrolo[2,3-b]pyridine-3-carboxamide hydrochloride

Starting materials: 3-methoxypropylamine and methyl 5-[4-(2-morpholin-4-ylethoxy)phenyl]-1H-pyrrolo[2,3-b]pyridine-3-carboxylate, yield 56%: ¹H NMR (D₂O, 400 MHz) δ 8.35 (s, 2H), 7.91 (s, 1H), 7.55 (m, 2H), 7.07 (m, 2H), 4.43 (m, 2H), 4.12 (m, 2H), 3.92 (m, 2H), 3.68 (m, 2H), 3.62 (m, 2H), 3.55 (m, 4H), 3.39 (m, 2H), 3.33 (s, 3H), 1.85 (m, 2H); MS (TSP) m/z 439 (M⁺+1).

### Example 22

### N-(3-Methoxypropyl)-5-{4-[2-(4-methylpiperazin-1-yl)ethoxy]phenyl}-1H-pyrrolo[2,3-b]pyridine-3-carboxamide hydrochloride

Starting materials: 3-methoxypropylamine and methyl 5-{4-[2-(4-methylpiperazin-1-yl)ethoxy]phenyl}-1H-pyrrolo[2,3-b]pyridine-3-carboxylate, yield 32%: ¹H NMR (D₂O, 400 MHz) δ 8.57 (m, 1H), 8.45 (m, 1H), 7.99 (s, 1H), 7.62 (m, 2H), 7.13 (m, 2H), 4.44 (m, 2H), 3.64 (m, 8H), 3.56 (m, 4H), 3.42 (m, 2H), 3.34 (s, 3H), 3.02 (s, 3H), 1.88 (m, 2H); MS (TSP) m/z 452 (M⁺+1).

### Example 23

### N-(3-Methoxyphenyl)-5-[4-(2-morpholin-4-ylethoxy)phenyl]-1H-pyrrolo[2,3-b]pyridine-3-carboxamide hydrochloride

Starting materials: (3-methoxyphenyl)amine and methyl 5-[4-(2-morpholin-4-ylethoxy)phenyl]-1H-pyrrolo[2,3-b]pyridine-3-carboxylate. The hydrochloride was triturated with diethyl ether and collected by centrifugation, yield 34%: ¹H NMR (DMSO-d₆, 400 MHz) δ 12.33 (d, J=2.5 Hz, 1H), 10.51 (m, 1H), 9.84 (s, 1H), 8.65 (d, J=2.3 Hz, 1H), 8.57 (d, J=2.3 Hz, 1H), 8.49 (d, J=3.0 Hz, 1H), 7.69 (m, 2H), 7.49 (m, 1H), 7.34 (m, 1H), 7.23 (t, J=8.1 Hz, 1H), 7.15 (m, 2H), 6.63 (m, 1H), 4.45 (m, 2H), 3.98 (m, 2H), 3.77 (m, 2H), 3.76 (s, 3H), 3.59 (m, 2H), 3.53 (m, 2H), 3.23 (m, 2H); MS (ES) m/z 473 (M⁺+1).

### Example 24

### N-(2-Methoxyphenyl)-5-[4-(2-morpholin-4-ylethoxy)phenyl]-1H-pyrrolo[2,3-b]pyridine-3-carboxamide hydrochloride

Starting materials: (2-methoxyphenyl)amine and methyl 5-[4-(2-morpholin-4-ylethoxy)phenyl]-1H-pyrrolo[2,3-b]pyridine-3-carboxylate, yield 19%: MS (ES) m/z 473 (M⁺+1).

### Example 25

### 5-{4-[2-(4-Methylpiperazin-1-yl)ethoxy]phenyl}-N-(2-morpholin-4-ylethyl)-1H-pyrrolo[2,3-b]pyridine-3-carboxamide hydrochloride

Starting materials: 4-(2-aminoethyl)morpholine and methyl 5-{4-[2-(4-methylpiperazin-1-yl)ethoxy]phenyl}-1H-pyrrolo[2,3-b]pyridine-3-carboxylate, yield 17%: ¹H NMR (DMSO-d₆, 400 MHz) δ 12.24 (d, J=2.5 Hz, 1H), 11.07 (m, 1H), 8.63 (m, 1H), 8.61 (d, J=2.3 Hz, 1H), 8.53 (d, J=2.3 Hz, 1H), 8.38 (d, J=2.8 Hz, 1H), 8.31 (s, 2H), 7.66 (m, 2H), 7.14 (m, 2H), 4.45 (m, 2H), 3.97 (m, 2H), 3.85 (m, 4H), 3.70 (m, 4H), 3.31 (m, 2H), 3.16 (s, 3H), 3.13 (m, 2H), 2.82 (m, 2H); MS (ES) m/z 493 (M⁺+1).

### Example 26

### 1-(4-Bromobenzyl)piperidine

To a solution of 4-bromobenzyl bromide (7.49 g, 30 mmol) in ethanol (100 mL) was added piperidine (3.3 mL, 33 mmol) and potassium carbonate (16.5 g, 119 mmol) and the mixture was stirred at reflux for 5 h. The mixture was filtered and the solvent was evaporated in vacuo. The crude material was treated with diethyl ether and the mixture was filtered. The solvent was evaporated in vacuo to give 6.57 gram (86% yield) of the title compound as a yellow oil: ¹H NMR (CDCl₃, 300 MHz) δ 7.41 (d, J=8.4 Hz, 2H), 7.19 (d, J=8.4 Hz, 2H), 3.40 (s, 2H), 2.33 (m, 4H), 1.56 (m, 4H), 1.50-1.38 (m, 2H).

### Example 27

### Methyl 5-[4-(piperidin-1-ylsulfonyl)phenyl]-1H-pyrrolo[2,3-b]pyridine-3-carboxylate

The title compound was synthesized as described for Example 7. Starting materials: 1-[(4-bromophenyl)sulfonyl]piperidine and methyl 5-bromo-1H-pyrrolo[2,3-b]pyridine-3-carboxylate, yield 81%: ¹H NMR (CDCl₃, 400 MHz) δ 8.71 (s, 1H), 8.63 (m, 1H), 8.09 (s, 1H), 7.88 (d, J=8.3 Hz, 2H), 7.80 (d, J=8.3 Hz, 2H), 3.96 (s, 3H), 3.06 (m, 4H), 1.67 (m, 4H), 1.45 (m, 3H); MS (TSP) m/z 400 (M⁺+1).

### Example 28

### 5-[4-(2-Morpholin-4-ylethoxy)phenyl]-N-pyridin-3-yl-1H-pyrrolo[2,3-b]pyridine-3-carboxamide hydrochloride

Trimethylaluminum (0.3 mL, 2.0 M in toluene, 0.6 mmol) was added to a cooled (0° C.) mixture of pyridin-3-amine (0.019 g, 0.19 mmol) and methyl 5-[4-(2-morpholin-4-ylethoxy)phenyl]-1H-pyrrolo[2,3-b]pyridine-3-carboxylate (0.025 g, 0.06 mmol), under an atmosphere of nitrogen. The resulting mixture was refluxed for 2 h. The mixture was allowed to cool to room temperature, saturated aqueous sodium hydrogen carbonate (2 mL) was added and the resulting mixture was stirred for 30 min. The mixture was centrifuged, the solvents were removed, the resulting residue was washed with toluene, the mixture centrifuged, and the toluene removed. The resulting solid was treated with methanol and the formed suspension was stirred for 45 min, centrifuged, and the methanol phase was evaporated. Chloroform was added and undissolved material was removed by filtration. The solution was treated with hydrochloric acid (4 M, in diethyl ether), the formed precipitate was removed by filtration, and dried in vacuo at 70° C. to give un-pure hydrochloride. The hydrochloride was portioned between aqueous sodium hydroxide (2 M) and dichloromethane, the organic phase was dried over sodium sulfate, and the solvent was evaporated. The formed base was purified on a silica gel column using methanol, as the eluent, and the solvent was evaporated. Chloroform was added and the solution was treated with hydrochloric acid (4 M, in diethyl ether), the formed precipitate was removed by filtration, and dried in vacuo at 70° C. Re-crystallization from methanol gave 1 mg (3.4% yield) of the title compound as solid: MS (ES) m/z 444 (M⁺+1).

Examples 29-32 were synthesized as described for Example 28.

### Example 29

### N-[2-(Methylsulfonyl)ethyl]-5-[4-(piperidin-1-ylsulfonyl)phenyl]-1H-pyrrolo[2,3-b]pyridine-3-carboxamide hydrochloride

Starting material: 2-(methylsulfonyl)ethanamine and methyl 5-[4-(piperidin-1-ylsulfonyl)phenyl]-1H-pyrrolo[2,3-b]pyridine-3-carboxylate. The evaporated methanol phase was purified on a silica gel column using gradient of dichloromethane/methanol (0-5% methanol), as the eluent. The combined fractions were evaporated and dried in vacuo at 80° C. Chloroform was added and the solution was treated with hydrochloric acid (4 M, in diethyl ether), the formed precipitate was removed by filtration, and dried in vacuo at 80° C. to give 1.3 mg (2.4% yield) of the title compound as a solid: MS (DI/EI) m/z 491 (M⁺)

### Example 30

### 5-[4-(Morpholin-4-ylmethyl)phenyl]-N-[2-(2-thienyl)ethyl]-1H-pyrrolo[2,3-b]pyridine-3-carboxamide hydrochloride

Starting material: 2-(2-thienyl)ethanamine and methyl 5-[4-(morpholin-4-ylmethyl)phenyl]-1H-pyrrolo[2,3-b]pyridine-3-carboxylate, yield 43%: ¹H NMR (CD₃OD, 400 MHz) δ 9.07 (d, J=1.8 Hz, 1H), 8.73 (d, J=1.8 Hz, 1H), 8.20 (s, 1H), 7.91 (d. J=8.3 Hz, 2H), 7.73 (d, J=8.1 Hz, 2H), 7.21 (dd, J=4.8, 1.5 Hz, 1H), 6.94 (m, 2H), 4.47 (s, 2H), 4.08 (dd, J=13.0, 3.2 Hz, 2H), 3.78 (m, 2H), 3.68 (t, J=7.2 Hz, 2H), 3.44 (d, J=12.9 Hz, 2H), 3.27 (d, J=3.3 Hz, 2H), 3.19 (t, J=7.1 Hz, 2H).

### Example 31

### N-Methyl-5-[4-[(4-methylpiperazin-1-yl)sulfonyl]-3-(trifluoromethoxy)phenyl]-1H-pyrrolo[2,3-b]pyridine-3-carboxamide hydrochloride

Starting material: methanamine and methyl 5-[4-[(4-methylpiperazin-1-yl)sulfonyl]-3-(trifluoromethoxy)phenyl]-1H-pyrrolo[2,3-b]pyridine-3-carboxylate, yield 33%: ¹H NMR (DMSO-d₆, 400 MHz) δ 12.36 (d, J=2.0 Hz, 1H), 11.00 (s, 1H), 8.76 (d, J=2.3 Hz, 1H), 8.70 (d, J=2.3 Hz, 1H), 8.21 (d, J=2.8 Hz, 1H), 8.12 (d, J=4.3 Hz, 1 H), 8.04 (m, 2H), 7.93 (s, 1H), 3.86 (d, J=9.5 Hz, 2H), 3.49 (m, 2H), 3.12 (m, 4H), 2.80 (d, J=4.5 Hz, 3H), 2.77 (s, 3H).

### Example 32

### N-Methyl-5-{4-[(4-methylpiperazin-1-yl)sulfonyl]phenyl}-1H-pyrrolo[2,3-b]pyridine-3-carboxamide hydrochloride

Starting material: methanamine and methyl 5-{4-[(4-methylpiperazin-1-yl)sulfonyl]phenyl}-1H-pyrrolo[2,3-b]pyridine-3-carboxylate, yield 43%: ¹H NMR (DMSO-d₆, 400 MHz) δ 12.30 (d, J=2.0 Hz, 1H), 10.58 (s, 1H), 8.77 (d, J=2.3 Hz, 1H), 8.68 (d, J=2.3 Hz, 1H), 8.19 (d, J=2.8 Hz, 1H), 8.09 (m, 1H), 8.06 (d, J=8.3 Hz, 2H), 7.98 (d, J=8.3 Hz, 2H), 3.83 (d, J=12.3 Hz, 2H), 3.48 (m, overlap with water in DMSO), 3.17 (m, 2H), 2.80 (d, J=3.5 Hz, 3H), 2.73 (m, 5H).

### Example 33

### 5-Bromo-N-(2-morpholin-4-ylethyl)-1H-pyrrolo[2,3-b]pyridine-3-carboxamide

Trimethylaluminum (1.8 mL, 2.0 M in toluene, 3.9 mmol) was added to a cooled (0° C.) mixture of 2-morpholin-4-ylethanamine (0.255 g, 1.96 mmol) and methyl 5-bromo-1H-pyrrolo[2,3-b]pyridine-3-carboxylate (0.100 g, 0.39 mmol), under an atmosphere of argon. The resulting mixture was refluxed for 30 min. The mixture was cooled to 5° C. and then quenched under stirring by addition of acetonitrile (4 mL) followed by methanol (3 mL). After 15 min the mixture was centrifuged, the solvents were evaporated. The residue was purified on a silica gel column using chloroform/methanol, (85:15), as the eluent, to give 90 mg (65% yield) of the title compound: ¹H NMR (DMSO-d₆, 400 MHz) δ 12.32 (br s, 1H), 8.58 (d, J=2.3 Hz, 1H), 8.34 (d, J=2.3 Hz, 1H), 8.17 (s, 1H), 8.01 (t, J=5.7 Hz, 1H), 3.58 (t, J=4.7 Hz, 4H), 3.38 (q, J=6.6 Hz, 2H), 2.46 (t, J=7.1 Hz, 2H), 2.42 (m, 4H).

Examples 34-36 were synthesized as described for Example 33.

### Example 34

### 5-Bromo-N-[2-(2-thienyl)ethyl]-1H-pyrrolo[2,3-b]pyridine-3-carboxamide

Starting material: 2-(2-thienyl)ethanamine and methyl 5-bromo-1H-pyrrolo[2,3-b]pyridine-3-carboxylate, yield 37%: ¹H NMR (DMSO-d₆, 400 MHz) δ 8.57 (d, J=2.3 Hz, 1H), 8.334 (d, J=2.3 Hz, 1H), 8.25 (t, J=5.6, 1H), 8.18 (s, 1H), 7.33 (dd, J=5.2, 1.1 Hz, 1H), 6.96 (m, 1H), 6.92 (m, 1H), 3.50 (q, J=6.7 Hz, 2H), 3.07 (t, J=7.2 Hz, 2H).

### Example 35

### 5-Bromo-N-methyl-1H-pyrrolo[2,3-b]pyridine-3-carboxamide

Starting material: methanamine and methyl 5-bromo-1H-pyrrolo[2,3-b]pyridine-3-carboxylate, yield 37%: ¹H NMR (DMSO-d₆, 400 MHz) δ 12.30 (s, 1H), 8.58 (d, J=2.3 Hz, 1H), 8.34 (d, J=2.3 Hz, 1H), 8.14 (s, 1H), 8.03 (d, J=4.6 Hz, 1H), 2.78 (d, J=4.6 Hz, 3H).

### Example 36

### 5-Bromo-N-pentyl-1H-pyrrolo[2,3-b]pyridine-3-carboxamide

Starting material: pentan-1-amine and methyl 5-bromo-1H-pyrrolo[2,3-b]pyridine-3-carboxylate, yield 37%: ¹H NMR (DMSO-d₆, 400 MHz) δ 12.29 (s, 1H), 8.58 (d, J=2.3 Hz, 1H), 8.33 (d, J=2.3 Hz, 1H), 8.19 (s, 1H), 8.04 (t, J=5.7 Hz, 1H), 3.24 (m, 2H), 1.52 (p, J=7.1 Hz, 2H), 1.31 (m, 4H), 0.88 (m, 3H).

### Example 37

### N-Methyl-5-[4-(piperidin-1-ylsulfonyl)phenyl]-1H-pyrrolo[2,3-b]pyridine-3-carboxamide hydrochloride

A mixture of [4-(piperidin-1-ylsulfonyl)phenyl]boronic acid (0.061 g, 0.23 mmol), 5-bromo-N-methyl-1H-pyrrolo[2,3-b]pyridine-3-carboxamide, (0.040 g, 0.16 mmol), aqueous sodium carbonate (aq. 0.23 mL, 2 M, 0.46 mmol) and [1,1′bis(diphenylphosphino)ferrocene]palladium(II) chloride dichloromethane adduct (0.026 g, 0.032 mmol) in anhydrous N,N-dimethylformamide (3 mL) was heated in a microwave reactor at 140° C. for 10 min. The mixture was evaporated and dried in vacuo at 60° C. The residue was purified on a silica gel column using chloroform/methanol, (85:15), as the eluent. The residue was dissolved in chloroform/methanol (9:1) and the solution was treated with hydrochloric acid (4 M, in diethyl ether). The formed precipitate was removed by filtration, and dried in vacuo at 60° C. to afford 35 mg (50% yield) of the title compound as solid: ¹H NMR (DMSO-d₆, 400 MHz) δ 12.27 (s, 1H), 8.75 (d, J=2.3 Hz, 1H), 8.66 (d, J=2.3 Hz, 1H), 8.17 (d, J=2.8 Hz, 1H), 8.06 (m, 1H), 7.99 (d, J=8.3 Hz, 2H), 7.83 (d, J=8.3 Hz, 2H), 2.94 (t, J=5.3 Hz, 4H), 2.80 (d, J=3.5 Hz, 3H), 1.55 (m, 4H), 1.38 (m, 2H).

Examples 38-41 were synthesized as described for Example 37.

### Example 38

### N-(2-Morpholin-4-ylethyl)-5-(4-morpholin-4-ylphenyl)-1H-pyrrolo[2,3-b]pyridine-3-carboxamide hydrochloride

Starting material: (4-morpholin-4-ylphenyl)boronic acid and 5-bromo-N-(2-morpholin-4-ylethyl)-1H-pyrrolo[2,3-b]pyridine-3-carboxamide, yield 30%: ¹H NMR on the base (CDCl₃, 400 MHz) δ 9.48 (br s, 1H), 8.59 (d, J=2.0 Hz, 1H), 8.49 (s, 1H), 7.95 (s, 1H), 7.59 (d, J=8.8 Hz, 2H), 7.03 (d, J=8.6 Hz, 2H), 3.91 (t, J=4.8 Hz, 4H), 3.76 (br s, 4H), 3.65 (m, 2H), 3.24 (t, J=4.9 Hz, 4H), 2.70 (m, 2H), 2.60 (m, 4H).

### Example 39

### N-(2-Morpholin-4-ylethyl)-5-[4-(pyrrolidin-1-ylcarbonyl)phenyl]-1H-pyrrolo[2,3-b]pyridine-3-carboxamide hydrochloride

Starting material: [4-(pyrrolidin-1-ylcarbonyl)phenyl]boronic acid and 5-bromo-N-(2-morpholin-4-ylethyl)-1H-pyrrolo[2,3-b]pyridine-3-carboxamide, yield 47%: ¹H NMR (DMSO-d₆, 400 MHz) δ 12.33 (d, J=2.5 Hz, 1H), 10.50 (br s, 1H), 8.71 (d, J=2.3 Hz, 1H), 8.64 (d, J=2.3 Hz, 1H), 8.54 (t, J=5.6 Hz, 1H), 8.34 (d, J=2.8 Hz, 1H), 7.78 (d, J=8.3 Hz, 2H), 7.66 (d, J=8.3 Hz, 2H), 4.00 (d, J=11.1 Hz, 2H), 3.80 (t, J=11.6 Hz, 2H), 3.70 (q, J=5.8 Hz, 2H), 3.58 (d, J=12.1 Hz, 2H), 3.49 (m, overlap with water in DMSO), 3.34 (m, 2H), 3.16 (m, 2H), 1.87 (m, 4H).

### Example 40

### 5-[4-Morpholin-4-ylcarbonyl)phenyl]-N-(2-morpholin-4-ylethyl)-1H-pyrrolo[2,3-b]pyridine-3-carboxamide hydrochloride

Starting material: [4-(morpholin-4-ylcarbonyl)phenyl]boronic acid and 5-bromo-N-(2-morpholin-4-ylethyl)-1H-pyrrolo[2,3-b]pyridine-3-carboxamide, yield 52%: ¹H NMR (DMSO-d₆, 400 MHz) δ 12.33 (d, J=2.5 Hz, 1H), 10.53 (br s, 1H), 8.70 (d, J=2.3 Hz, 1H), 8.63 (d, J=2.3 Hz, 1H), 8.54 (t, J=5.6 Hz, 1H), 8.34 (d, J=2.8 Hz, 1H), 7.79 (d, J=8.3 Hz, 2H), 7.55 (d, J=8.3 Hz, 2H), 3.99 (d, J=10.9 Hz, 2H), 3.79 (t, J=11.5 Hz, 2H), 3.69 (m, 2H), 3.59 (m, overlap with water in DMSO), 3.33 (q, J=5.9 Hz, 2H), 3.14 (m, 2H).

### Example 41

### 5-[4-(Morpholin-4-ylmethyl)phenyl]-N-pentyl-1H-pyrrolo[2,3-b]pyridine-3-carboxamide hydrochloride

Starting material: 4-[4-(4,4,5,5-tetramethyl-1,3,2-dioxaborolan-2-yl)benzyl]morpholine and 5-bromo-N-pentyl-1H-pyrrolo[2,3-b]pyridine-3-carboxamide, yield 13%: ¹H NMR (DMSO-d₆, 400 MHz) δ 12.19 (d, J=2.3 Hz, 1H), 10.73 (s, 1H), 8.70 (d, J=2.0 Hz, 1H), 8.61 (d, J=2.3 Hz, 1H), 8.20 (d, J=2.8 Hz, 1H), 8.06 (t, J=5.6 Hz, 1H), 7.83 (d, J=8.1 Hz, 2H), 7.70 (d, J=8.3 Hz, 2H), 4.40 (d, J=5.0 Hz, 2H), 3.96 (d, J=12.1 Hz, 2H), 3.27 (m, 4H), 3.13 (m, 2H), 1.54 (p, J=7.1 Hz, 2H), 1.32 (m, 4H), 0.89 (t, J=6.8 Hz, 3H).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt/solvate (active ingredient) is in association with a pharmaceutically acceptable excipient, diluent or carrier. Dependent on the mode of administration, the pharmaceutical composition may comprise from 0.05 to 99% w (percent by weight), for example from 0.10 to 50% w, of active ingredient, all percentages by weight being based on total composition.

An excipient, diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt, solvate or solvate of salt thereof, as hereinbefore defined, with a pharmaceutically acceptable excipient, diluent or carrier.

An example of a pharmaceutical composition of the invention is an injectable solution containing a compound of the invention, or a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

Liquid solution comprising a compound of formula I, or a salt thereof, dissolved in water.

| Solution | mg/mL |
|---|---|
| Compound X | 5.0% w/v |
| Pure water | To 100% |

### Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders.

One embodiment of the invention relates to the prevention and/or treatment of dementia and Alzheimer's Disease.

Another embodiment of the invention relates to the prevention and/or treatment of bone-related disorders.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, including man in need of such treatment and/or prevention a therapeutically effective amount of a compound of formula I, as hereinbefore defined.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β used to calculate the inhibition constants (Kᵢ) of the various compounds, was 20 μM.

The following abbreviations have been used:

MOPS Morpholinepropanesulfonic acid

EDTA Ethylenediaminetetraacetic acid

BSA Bovin Serum Albumin

ATP Adenosine Triphosphate

SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

1. A compound of the formula I: 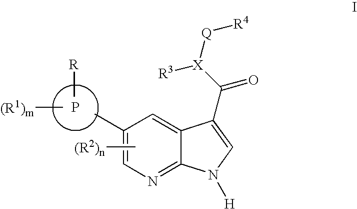
wherein:
P is phenyl or a 5- or 6-membered heteroaromatic ring containing one or more heteroatoms selected from N, O or S and said phenyl or 5- or 6-membered heteroaromatic ring may optionally be fused with a 5- or 6-membered saturated, partially saturated or unsaturated ring containing one or more atoms selected from C, N, O or S;
Q is independently selected from C₁₋₆alkyl, C₂₋₆alkenyl or C₂₋₆alkynyl, or Q is absent;
X is independently selected from N or O and when X is O, Q and R⁴ are absent;
R is independently selected from hydrogen, CN, NO₂, OH, NH₂, COOH, CONH₂, COCH₃, halogen, C₁₋₆alkyl, C₁₋₆alkoxy, fluoromethyl, difluoromethyl, trifluoromethyl, fluoromethoxy, difluoromethoxy, trifluoromethoxy;
R¹ is independently selected from C₁₋₆alkylC₃₋₆cycloalkyl, OR⁵, SR⁵, NR⁶R⁷, CO₂R⁵, COR⁵, (SO₂)R⁵, (SO)R⁵, (SO₂)NR⁶R⁷, NR⁸(SO₂)R⁵, CONR⁶R⁷, NR⁸COR⁵, NR⁸CONR⁶R⁷, NR⁸CO₂R⁵, (SO)NR⁶R⁷, NR⁸(SO)R⁵, C₁₋₆alkylOR⁵, C₁₋₆alkylSR⁵, C₁₋₆alkylNR⁶R⁷, C₁₋₆alkylCO₂R⁵, C₁₋₆alkylCOR⁵, C₁₋₆alkyl(SO₂)R⁵, C₁₋₆alkyl(SO)R⁵, C₁₋₆alkyl(SO₂)NR⁶R⁷, C₁₋₆alkylNR⁸(SO₂)R⁵, C₁₋₆alkylCONR⁶R⁷, C₁₋₆alkylNR⁸COR⁵, C₁₋₆alkylNR⁸CONR⁶R⁷, C₁₋₆alkylNR⁸CO₂R⁵, C₁₋₆alkyl(SO)NR⁶R⁷ or C₁₋₆alkylNR⁸(SO)R⁵; and wherein said C₁₋₆alkyl group is optionally substituted by one or more A;
R² is independently selected from hydrogen, C₁₋₆alkyl, CN, nitro, halogen, OR¹³, fluoromethyl, difluoromethyl, trifluoromethyl, fluoromethoxy, difluoromethoxy and trifluoromethoxy;
R³ is independently selected from hydrogen, C₁₋₆alkyl, C₂₋₆alkenyl, C₂₋₆alkynyl and C₀₋₆alkylC₃₋₆cycloalkyl, wherein said C₁₋₆alkyl, C₂₋₆alkenyl, C₂₋₆alkynyl and C₀₋₆alkylC₃₋₆cycloalkyl group is optionally substituted by one or more A;
R⁴ is independently selected from hydrogen, halogen, nitro, CHO, CN, OC₁₋₆alkylCN, OR¹³, OC₁₋₆alkylOR¹³, fluoromethyl, difluoromethyl, trifluoromethyl, fluoromethoxy, difluoromethoxy, trifluoromethoxy, NR¹³R¹⁴, OC₁₋₆alkylNR¹³R¹⁴, NR¹³OR¹⁴, CO₂R¹³, OC₁₋₆alkylCO₂R¹³, CONR¹³R¹⁴, OC₁₋₆alkylCONR¹³R¹⁴, OC₁₋₆alkylNR¹³(CO)R¹⁴, NR¹³(CO)R¹⁴, O(CO)NR¹³R¹⁴, NR¹³(CO)OR¹⁴, NR¹³(CO)NR¹³R¹⁴, O(CO)OR¹³O(CO)R¹³, COR¹³, OC₁₋₆alkylCOR¹³NR¹³(CO)(CO)R¹⁴, NR¹³(CO)(CO)NR¹³R¹⁴, SR¹³, (SO₂)NR¹⁴R¹³, OC₁₋₆alkylNR¹³(SO₂)R¹⁴, NR⁵(SO₂)R¹³, OC₀₋₆alkyl(SO₂)NR¹³R¹⁴, (SO)NR¹³R¹⁴, OC₁₋₆alkyl(SO)NR¹³R¹⁴, SO₃R¹³, NR⁵(SO₂)NR¹³R¹⁴, NR¹³(SO)R¹⁴, OC₁₋₆alkylNR¹³(SO)R¹⁴, OC₀₋₆alkylSO₂R¹³, SO₂R¹³, SOR¹³, C₃₋₆cycloalkyl, aryl, a 5- or 6-membered heteroaromatic ring containing one or more heteroatoms independently selected from N, O, or S, a 5- or 6-membered heterocyclic ring containing one or more heteroatoms independently selected from N, O, or S, which heterocyclic group may be saturated or unsaturated, or R⁴ may be absent, and wherein any said C₃₋₆cycloalkyl, aryl, 5- or 6-membered heteroaromatic ring or a 5- or 6-membered heterocyclic ring is optionally substituted by one or more A, or R⁴ may be absent;
R⁵ is independently selected from C₃₋₆cycloalkyl, C₁₋₆alkylC₃₋₆cycloalkyl, C₂₋₆alkylNR⁹R¹⁰, C₂₋₆alkylOC₂₋₆alkylNR⁹R¹⁰, C₂₋₆alkyl(SO₂)NR⁹R¹⁰, C₂₋₆alkyl(SO)NR⁹R¹⁰, C₂₋₆alkylNR¹¹(SO)R¹², C₂₋₆alkylNR⁹(SO₂)R¹⁰, C₂₋₆alkyl(SO₂)C₂₋₆alkylNR⁹R¹⁰, C₂₋₆alkyl(SO)C₂₋₆alkylNR⁹R¹⁰, C₂₋₆alkylSC₂₋₆alkylNR¹¹R¹², C₂₋₆alkylCONR⁹R¹⁰, C₂₋₆alkylNR⁹COR¹⁰, heteroaryl or C₁₋₆alkylheteroaryl wherein any C₂₋₆alkyl or heteroaryl is optionally substituted by one or more A;
R⁶ is independently selected from C₂₋₆alkenyl, C₂₋₆alkynyl, C₀₋₆alkylC₃₋₆cycloalkyl, C₁₋₆alkylNR⁹R¹⁰, C₁₋₆alkylOC₁₋₆alkylNR⁹R¹⁰, C₀₋₆alkyl(SO₂)NR⁹R¹⁰, C₁₋₆alkyl(SO)NR⁹R¹⁰, C₁₋₆alkylNR¹¹(SO)R¹², C₁₋₆alkylNR⁹(SO₂)R¹⁰, C₀₋₆alkyl(SO₂)C₁₋₆alkylNR⁹R¹⁰, C₁₋₆alkyl(SO)C₁₋₆alkylNR⁹R¹⁰, C₂₋₆alkylSC₂₋₆alkylNR¹¹R¹², C₁₋₆alkylCONR⁹R¹⁰, C₀₋₆alkylNR⁹COR¹⁰, C₁₋₆alkylaryl or C₀₋₆alkylheteroaryl wherein any C₁₋₆alkyl, C₂₋₆alkenyl, C₂₋₆alkynyl, C₀₋₆alkylC₃₋₆cycloalkyl, C₁₋₆alkylaryl, C₀₋₆alkylheteroaryl is optionally substituted by one or more A;
R⁷ is independently selected from hydrogen and C₁₋₆alkyl;
R⁶ and R⁷ may together form a 5- or 6-membered heterocyclic ring containing one or more heteroatoms selected from N, O or S, wherein said heterocyclic ring is optionally substituted by one or more A;
R⁸ is independently selected from hydrogen, C₁₋₆alkyl, C₂₋₆alkenyl, C₂₋₆alkynyl, C₀₋₆alkylC₃₋₆cycloalkyl;
R⁹ is independently selected from C₁₋₆alkyl-B, C₂₋₆alkenyl, C₂₋₆alkynyl, C₃₋₆cycloalkyl, C₁₋₆alkylC₃₋₆cycloalkyl, aryl, C₁₋₆alkylaryl, heteroaryl, C₁₋₆alkylheteroaryl wherein any C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;
R¹⁰ is independently selected from hydrogen and C₁₋₆alkyl;
R⁹ and R¹⁰ may together form a 5- or 6-membered heterocyclic ring containing one or more heteroatoms selected from N, O or S, wherein said heterocyclic ring is optionally substituted by one or more A;
R¹¹ and R¹² are independently selected from hydrogen, C₁₋₆alkyl, C₂₋₆alkenyl, C₂₋₆alkynyl, C₃₋₆cycloalkyl, C₁₋₆alkylC₃₋₆cycloalkyl, aryl, C₁₋₆alkylaryl, heteroaryl, C₁₋₆alkylheteroaryl, wherein any C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;
R¹¹ and R¹² may together form a 5- or 6-membered heterocyclic ring containing one or more heteroatoms selected from N, O or S, wherein said heterocyclic ring is optionally substituted by one or more A;
R¹³ and R¹⁴ are independently selected from hydrogen, C₁₋₆alkyl, C₂₋₆alkenyl, C₂₋₆alkynyl, C₃₋₆cycloalkyl, C₁₋₆alkylC₃₋₆cycloalkyl, C₁₋₆alkylNR¹⁵R¹⁶, aryl, C₁₋₆alkylaryl, heteroaryl, C₁₋₆alkylheteroaryl, wherein any said C₃₋₆cycloalkyl, C₂₋₆alkenyl, C₂₋₆alkynyl, C₁₋₆alkylC₃₋₆cycloalkyl, aryl, C₁₋₆alkylaryl, heteroaryl, C₁₋₆alkylheteroaryl is optionally substituted by one or more A;
R¹³ and R¹⁴ may together form a 5- or 6-membered heterocyclic ring containing one or more heteroatoms selected from N, O or S, wherein said heterocyclic ring is optionally substituted by one or more A;
R¹⁵ and R¹⁶ are independently selected from hydrogen and C₁₋₆alkyl;
R¹⁵ and R¹⁶ may together form a 5- or 6-membered heterocyclic ring containing one or more heteroatoms selected from N, O or S, wherein the said heterocyclic ring is optionally substituted by one or more A;
m is 1;
n is 1 or 2;
A is selected from: halogen, nitro, oxo (═O), CHO, CN, OR¹⁵, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR¹⁵R¹⁶, OC₁₋₆alkylNR¹⁵R¹⁶, CO₂R¹⁵, CONR¹⁵R¹⁶, NR¹⁵(CO)R¹⁶, O(CO)R¹⁵, COR¹⁵, SR¹⁵, (SO₂)NR¹⁵R¹⁶, (SO)NR¹⁵R¹⁶, SO₃R¹⁵, SO₂R¹⁵ or SOR¹⁵;
B is nitro, oxo (═O), CHO, CN, OR¹⁵, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR¹⁵R¹⁶, OC₁₋₆alkylNR¹⁵R¹⁶, CO₂R¹⁵, CONR¹⁵R¹⁶, NR¹⁵(CO)R¹⁶, O(CO)R¹⁵, COR¹⁵, SR¹⁵, (SO₂)NR¹⁵R¹⁶, (SO)NR¹⁵R¹⁶, SO₃R¹⁵, SO₂R¹⁵ or SOR¹⁵;
as a free base or a pharmaceutically acceptable salt, solvate or solvate of a salt thereof.

2. A compound according to claim 1, wherein P is phenyl.

3. A compound according to claim 1, wherein R is selected from hydrogen, halogen, C₁₋₆alkyl, trifluoromethyl and trifluoromethoxy.

4. A compound according to claim 3, wherein R is selected from hydrogen and trifluoromethoxy.

5. A compound according to claim 1, wherein R¹ is selected from NR⁶R⁷, C₁₋₆alkylNR⁶R⁷, CONR⁶R⁷, (SO₂)NR⁶R⁷ and OR⁵.

6. A compound according to claim 1, wherein R² is hydrogen.

7. A compound according to claim 1, wherein R³ is hydrogen or C₁₋₆alkyl.

8. A compound according to claim 7, wherein R³ is hydrogen.

9. A compound according to claim 1, wherein R⁴ is independently selected from OR¹³, NR¹³R¹⁴, CN, SOR¹³, SO₂R¹³, and a 5- or 6-membered heteroaromatic ring containing one or more heteroatoms independently selected from N, O, or S, or a 5- or 6-membered heterocyclic ring containing one or more heteroatoms independently selected from N, O, or S which heterocyclic group may be saturated or unsaturated, and wherein said 5- or 6-membered heteroaromatic ring or said 5- or 6-membered heterocyclic ring is optionally substituted by one or more A.

10. A compound according to claim 1, wherein R¹³ and R¹⁴ together form a 5- or 6-membered heterocyclic ring containing one or more heteroatoms selected from N, O or S, wherein said heterocyclic ring is optionally substituted by one or more A.

11. A compound according to claim 9 or 10, wherein said A is selected from C₁₋₆alkyl, OR¹⁵ and SO₂R¹⁵.

12. A compound according to claim 1, wherein Q is C₁₋₆alkyl, or Q is absent.

13. A compound according to claim 1, wherein X is N.

14. A compound according to claim 1 wherein R is selected from hydrogen, and trifluoromethoxy; R¹ is selected from NR⁶R⁷, C₁₋₆alkylNR⁶R⁷, CONR⁶R⁷, (SO₂)NR⁶R⁷, and OR⁵; R² and R³ is hydrogen; R⁴ is independently selected from OR¹³, NR¹³R¹⁴, SO₂R¹³, and a 5- or 6-membered heteroaromatic ring containing one or more heteroatoms independently selected from N, O, or S, a 5- or 6-membered heterocyclic ring containing one or more heteroatoms independently selected from N, O, or S which heterocyclic group may be saturated or unsaturated, and wherein said 5- or 6-membered heteroaromatic ring or said 5- or 6-membered heterocyclic ring is optionally substituted by one or more A; R¹³ and R¹⁴ are independently selected from hydrogen, and C₁₋₆alkyl; R¹³ and R¹⁴ together form a 5- or 6-membered heterocyclic ring containing one or more heteroatoms selected from N, O or S, wherein the said heterocyclic ring is optionally substituted by a group selected from A; R¹⁵ is C₁₋₆alkyl; Q is C₁₋₆alkyl, or Q is absent; A is C₁₋₆alkyl, SO₃R¹⁵, or OR¹⁵.

15. A compound selected from:
N-(3-Methoxypropyl)-5-{4-[(4-methylpiperazin-1-yl)sulfonyl]phenyl}-1H-pyrrolo[2,3-b]pyridine-3-carboxamide hydrochloride;
5-{4-[(4-Methylpiperazin-1-yl)sulfonyl]phenyl}-N-pyridin-3-yl-1H-pyrrolo[2,3-b]pyridine-3-carboxamide hydrochloride;
N-(2-Methoxyethyl)-5-{4-[(4-methylpiperazin-1-yl)sulfonyl]phenyl}-1H-pyrrolo[2,3-b]pyridine-3-carboxamide hydrochloride;
N-(2-Methoxyethyl)-5-[4-[(4-methylpiperazin-1-yl)sulfonyl]-3-(trifluoromethoxy)phenyl]-1H-pyrrolo[2,3-b]pyridine-3-carboxamide hydrochloride;
N-(3-Methoxypropyl)-5-[4-[(4-methylpiperazin-1-yl)sulfonyl]-3-(trifluoromethoxy)phenyl]-1H-pyrrolo[2,3-b]pyridine-3-carboxamide hydrochloride;
5-[4-[(4-Methylpiperazin-1-yl)sulfonyl]-3-(trifluoromethoxy)phenyl]-N-pyridin-3-yl-1H-pyrrolo[2,3-b]pyridine-3-carboxamide hydrochloride;
5-{4-[(4-methylpiperazin-1-yl)sulfonyl]phenyl}-N-[2-(methylsulfonyl)phenyl]-1H-pyrrolo[2,3-b]pyridine-3-carboxamide hydrochloride;
N-(2-Methoxyethyl)-5-[4-(morpholin-4-ylmethyl)phenyl]-1H-pyrrolo[2,3-b]pyridine-3-carboxamide hydrochloride;
N-(3-Methoxypropyl)-5-[4-(piperidin-1-ylmethyl)phenyl]-1H-pyrrolo[2,3-b]pyridine-3-carboxamide hydrochloride;
5-{4-[(4-Methylpiperazin-1-yl)methyl]phenyl}-N-[2-(methylsulfonyl)phenyl]-1H-pyrrolo[2,3-b]pyridine-3-carboxamide hydrochloride;
5-{4-[2-(4-Methylpiperazin-1-yl)ethoxy]phenyl}-N-pyridin-3-yl-1H-pyrrolo[2,3-b]pyridine-3-carboxamide hydrochloride;
N-(3-methoxypropyl)-5-[4-(2-morpholin-4-ylethoxy)phenyl]-1H-pyrrolo[2,3-b]pyridine-3-carboxamide hydrochloride;
N-(3-Methoxypropyl)-5-{4-[2-(4-methylpiperazin-1-yl)ethoxy]phenyl}-1H-pyrrolo[2,3-b]pyridine-3-carboxamide hydrochloride;
N-(3-Methoxyphenyl)-5-[4-(2-morpholin-4-ylethoxy)phenyl]-1H-pyrrolo[2,3-b]pyridine-3-carboxamide hydrochloride;
N-(2-Methoxyphenyl)-5-[4-(2-morpholin-4-ylethoxy)phenyl]-1H-pyrrolo[2,3-b]pyridine-3-carboxamide hydrochloride; and
5-{4-[2-(4-Methylpiperazin-1-yl)ethoxy]phenyl}-N-(2-morpholin-4-ylethyl)-1H-pyrrolo[2,3-b]pyridine-3-carboxamide hydrochloride;
5-[4-(2-Morpholin-4-ylethoxy)phenyl]-N-pyridin-3-yl-1H-pyrrolo[2,3-b]pyridine-3-carboxamide hydrochloride;
N-[2-(Methylsulfonyl)ethyl]-5-[4-(piperidin-1-ylsulfonyl)phenyl]-1H-pyrrolo[2,3-b]pyridine-3-carboxamide hydrochloride;
5-[4-(Morpholin-4-ylmethyl)phenyl]-N-[2-(2-thienyl)ethyl]-1H-pyrrolo[2,3-b]pyridine-3-carboxamide hydrochloride;
N-Methyl-5-[4-[(4-methylpiperazin-1-yl)sulfonyl]-3-(trifluoromethoxy)phenyl]-1H-pyrrolo[2,3-b]pyridine-3-carboxamide hydrochloride;
N-Methyl-5-{4-[(4-methylpiperazin-1-yl)sulfonyl]phenyl}-1H-pyrrolo[2,3-b]pyridine-3-carboxamide hydrochloride;
N-Methyl-5-[4-(piperidin-1-ylsulfonyl)phenyl]-1H-pyrrolo[2,3-b]pyridine-3-carboxamide hydrochloride;
N-(2-Morpholin-4-ylethyl)-5-(4-morpholin-4-ylphenyl)-1H-pyrrolo[2,3-b]pyridine-3-carboxamide hydrochloride;
N-(2-Morpholin-4-ylethyl)-5-[4-(pyrrolidin-1-ylcarbonyl)phenyl]-1H-pyrrolo[2,3-b]pyridine-3-carboxamide hydrochloride;
5-[4-(Morpholin-4-ylcarbonyl)phenyl]-N-(2-morpholin-4-ylethyl)-1H-pyrrolo[2,3-b]pyridine-3-carboxamide hydrochloride; or
5-[4-(Morpholin-4-ylmethyl)phenyl]-N-pentyl-1H-pyrrolo[2,3-b]pyridine-3-carboxamide hydrochloride.

16. A compound selected from:
Methyl 5-{4-[(4-methylpiperazin-1-yl)sulfonyl]phenyl}-1H-pyrrolo[2,3-b]pyridine-3-carboxylate;
Methyl 5-[4-[(4-methylpiperazin-1-yl)sulfonyl]-3-(trifluoromethoxy)phenyl]-1H-pyrrolo[2,3-b]pyridine-3-carboxylate;
Methyl 5-[4-(morpholin-4-ylmethyl)phenyl]-1H-pyrrolo[2,3-b]pyridine-3-carboxylate;
Methyl 5-{4-[(4-methylpiperazin-1-yl)methyl]phenyl}-1H-pyrrolo[2,3-b]pyridine-3-carboxylate;
Methyl 5-[4-(piperidin-1-ylmethyl)phenyl]-1H-pyrrolo[2,3-b]pyridine-3-carboxylate;
Methyl 5-[4-(2-morpholin-4-ylethoxy)phenyl]-1H-pyrrolo[2,3-b]pyridine-3-carboxylate;
Methyl 5-{4-[2-(4-methylpiperazin-1-yl)ethoxy]phenyl}-1H-pyrrolo[2,3-b]pyridine-3-carboxylate; or
Methyl 5-[4-(piperidin-1-ylsulfonyl)phenyl]-1H-pyrrolo[2,3-b]pyridine-3-carboxylate.

17. A pharmaceutical formulation comprising as an active ingredient a therapeutically effective amount of a compound according to claim 1 in association with a pharmaceutically acceptable excipient, carrier or diluent.

18-25. (canceled)

26. A method of prevention and/or treatment of conditions associated with glycogen synthase kinase-3, comprising administering to a mammal, including man in need of such prevention and/or treatment, a therapeutically effective amount of a compound of formula I as defined in claim 1.

27. A method of prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, comprising administering to a mammal, including man in need of such prevention and/or treatment, a therapeutically effective amount of a compound of formula I as defined in claim 1.

28. A method according to claim 27, wherein the disease is Alzheimer's Disease.

29. A method of prevention and/or treatment of amyotrophic lateral sclerosis, corticobasal degeneration, Down syndrome, Huntington's Disease, postencephalitic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication, comprising administering to a mammal, including man in need of such prevention and/or treatment, a therapeutically effective amount of a compound of formula I as defined in claim 1.

30. A method of prevention and/or treatment of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders, comprising administering to a mammal, including man in need of such prevention and/or treatment, a therapeutically effective amount of a compound of formula I as defined in claim 1.

31. A method of prevention and/or treatment of bone-related disorders, comprising administering to a mammal, including man in need of such prevention and/or treatment, a therapeutically effective amount of a compound of formula I as defined in claim 1.

32. A process for preparing a compound of formula I, wherein X, P, Q, R, R¹, R², R³, R⁴, R⁵, R⁶, R⁷, R⁸, R⁹, R¹⁰, R¹¹, R¹², R¹³, R¹⁴, R¹⁵ R¹⁶, A, m and n are, unless specified otherwise, defined as in formula I, comprising: 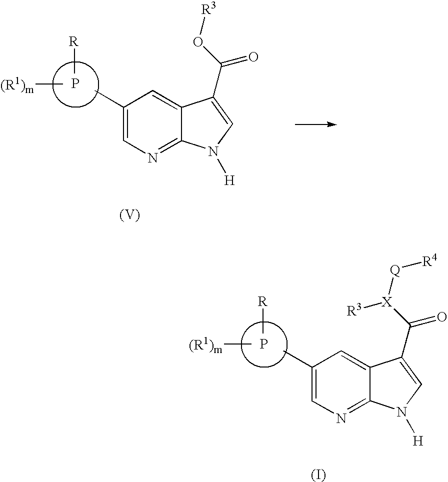
amidation of a compound of formula V, to obtain a compound of formula I, by treating a compound of formula V with an amine, HN(R³)QR⁴ preformed neat or by using a suitable solvent, optionally with the addition of a base or an acid.

33. A process for preparing a compound of formula I, wherein X, P, Q, R, R¹, R², R³, R⁴, R⁵, R⁶, R⁷, R⁸, R⁹, R¹⁰, R¹¹, R¹², R¹³, R¹⁴, R¹⁵ R¹⁶, A, m and n are, unless specified otherwise, defined as in formula I, comprising: 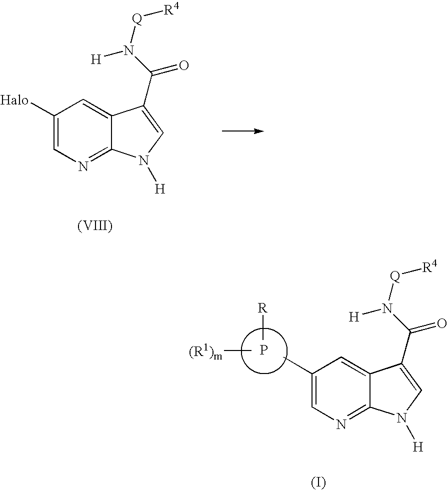
de-halogen coupling of a compound of formula VIII with an appropriate aryl species in a suitable solvent to give a compound of formula I.
